(19) United States
(12) Patent Application Publication
(10) Pub. No.: US 2026/0069633 A1
(43) Pub. Date: Mar. 12, 2026

(54) METHODS OF TREATING DISEASES ASSOCIATED WITH CELLULAR-ENERGY DEFICIENCY OR MITOCHONDRIAL DYSFUNCTION BY LOCOREGIONAL DELIVERY OF EXTRACELLULAR VESICLES THAT HAVE A CARGO WITH AN ENHANCED BIOENERGETIC PROFILE

(71) Applicant: THE BOARD OF TRUSTEES OF THE LELAND STANFORD JUNIOR UNIVERSITY, Stanford, CA (US)

(72) Inventors: Avnesh S. Thakor, Palo Alto, CA (US); Shashank Chetty, Redwood City, CA (US); Shobha Regmi, Redwood City, CA (US); Abantika Ganguly, Redwood City, CA (US)

(21) Appl. No.: 18/706,264

(22) PCT Filed: Nov. 1, 2022
(86) PCT No.: PCT/US2022/079066

§ 371 Date: Apr. 30, 2024

Related U.S. Application Data
(60) Provisional application No. 63/274,234, filed on Nov. 1, 2021.

(51) Int. Cl. A61K 35/28; A61P 11/00; C12N 13/00

(52) U.S. Cl. CPC A61K 35/28; A61P 11/00; C12N 13/00

(57) ABSTRACT

Methods for treating diseases associated with inflammation driven cellular-energy deficiency or mitochondrial dysfunction are provided. The methods utilize extracellular vesicles derived from mesenchymal stromal cells (MSCs) that have been stimulated with sound waves. The use of pFUS stimulation at low acoustic doses enhances the production and bioenergetic profile of extracellular vesicles from MSCs. The extracellular vesicles derived from MSCs that have been stimulated with sound waves can be used to reduce inflammation, restore the bioenergetic health of injured cells, and promote regeneration of injured tissue through the release of the extracellular vesicle cargo, which contains mitochondria-related products.

### BACKGROUND OF THE INVENTION

Mesenchymal stromal cell (MSC) therapy is a promising approach in regenerative medicine that has been shown to have significant potential for the repair of damaged tissue. MSCs are a heterogeneous population of cells found in various adult tissues. Due to their immunomodulatory and regenerative properties, as well as their ease of isolation and in vitro expansion, MSCs have been extensively explored as a platform for cellular therapy (Murphy et al. (2013) Exp. Mol. Med. 45:e54). Recently, it has become clear that the therapeutic effects of MSCs come not only from their ability to directly differentiate into new cells, but also by their release of soluble factors and extracellular vesicles (EVs) with regenerative properties (Caplan (2009) J. Pathol. 217(2):318-24). These molecules act through paracrine signaling to stimulate repair via anti-inflammatory, mitogenic, vasculotropic, and pro-survival pathways, which provide protection for surviving intrinsic epithelial cells and promote their proliferation (Morigi et al. (2014) Nephron Exp. Nephrol. 126(2):59, Lai et al. (2015) Semin. Cell Dev. Biol. 40:82-88). Since MSCs act via paracrine signaling, their proximity to the injured site is believed to be crucial for tissue regeneration. However, multiple studies have shown that when MSCs are injected intravenously, they are predominantly trapped in the lung microvasculature, in what is termed the pulmonary first pass effect (Schrepfer et al. (2007) Transplant Proc. 39(2):573-576, Santeramo et al. (2017) Stem Cells Transl Med. 6(5):1373-1384, Leibacher et al. (2016) Stem Cell Res. Ther. 7:7, Eggenhofer et al. (2012) Front. Immunol. 3:297, Fischer et al. (2009) Stem Cells Dev. 18(5):683-692). Consequently, instead of using whole cells, many studies have now begun investigating the use of purified EVs from MSCs as a cell-free therapy (Lv et al. (2018) J. Cell Mol. Med. 22(2):728-737), which are small enough to avoid pulmonary trapping (Phinney et al. (2017) Stem Cells 35(4):851-858).

MSC-derived EVs carry a cargo of regenerative molecules and have been shown to have a therapeutic effect in various animal models of disease (Cheng et al. (2017) Stem Cells Int. 2017:6305295, Ullah et al. (2020) Cells 9(4):937). Despite the growing interest in EVs for regenerative applications, there remains an unmet need to improve their therapeutic effect.

### SUMMARY OF THE INVENTION

Methods for treating diseases associated with cellular-energy deficiency or mitochondrial dysfunction are provided. The methods utilize extracellular vesicles (EVs) derived from mesenchymal stromal cells (MSCs) that have been treated with sound waves. The use of pulsed focused ultrasound (pFUS) stimulation at low acoustic doses enhances the production and bioenergetic profile of the EVs from MSCs. The pFUS stimulated MSC-derived EVs can be used to reduce inflammation, restore the bioenergetic health of injured cells, and promote regeneration of injured tissue through the release of the EV cargo, which contains mitochondria-related products.

The subject methods provide a controlled cell-free therapy with administration of EVs from MSCs stimulated with sound waves. Sound waves not only stimulate MSCs to produce EVs, but also increase the bioenergetic cargo within EVs as shown by the mitochondrial content of pFUS-stimulated EVs, which includes microRNA (miRNA), messenger RNA (mRNA), and proteins that are involved in mitochondrial biogenesis, function, and stimulation (see Example 1). The pFUS-stimulated MSC derived EVs (pFUS-MSC-EVs) can be delivered to patients by any suitable mode of administration, including, for example, without limitation, by intravenous injection or through directed local delivery at a target site (e.g., nebulization for delivery to the lungs; intra-arterial delivery for solid organs such as the kidneys, liver, heart, and brain; delivery to cerebrospinal fluid (CSF) for treatment of the brain; or percutaneous delivery for muscle, etc.)

Various diseases and conditions associated with cellular-energy deficiency or mitochondrial dysfunction can be treated by this method, including mitochondrial diseases, inflammatory diseases, hereditary diseases, infections, degenerative diseases, cardiovascular diseases, aging, infarction, chronic fatigue syndrome, and cancer.

In one aspect, a method of treating a subject for a disease or condition associated with cellular-energy deficiency or mitochondrial dysfunction is provided, the method comprising: stimulating a mesenchymal stromal cell with sound waves; and administering to the subject a therapeutically effective amount of extracellular vesicles derived from the mesenchymal stromal cell after said stimulating the mesenchymal stromal cell with the sound waves.

In certain embodiments, the sound waves are administered in an effective amount sufficient to increase levels in the extracellular vesicles of a mitochondrial microRNA (miRNA), a mitochondrial messenger RNA (mRNA), a mitochondrial protein, lipids, or a combination thereof, compared to the levels in extracellular vesicles produced by a reference mesenchymal stromal cell that is not stimulated with the sound waves. In some embodiments, stimulation of the MSC with sound waves results in production of extracellular vesicles having higher amounts of mitochondrial proteins such COX-IV, TOM20, Complex I, Complex II, Complex II and Complex IV, citrate synthase, HSP60, PGC-1α, SIRT1, SIRT2, SIRT3, MFN, OPA1, DRP1, TRPC, PMCA, RhoA1, Miro1, or mtHSP70. In some embodiments, stimulation of the MSC with sound waves results in production of extracellular vesicles having higher amounts of miRNAs involved in immunomodulation or metabolic health, such as mir-9-5p, miR-15a-5p, miR-22-3p, miR-224-3p, miR-144-3p, miR-146a-5p, miR-9-5p, miR-15a-5p, miR-16-5p, miR-18a-5p, miR-19b-3p, miR-20a-5p, miR-29a-3p, miR30a-5p, miR-30b-5p, miR-30e-5p, miR-34a-5p, miR-92a-3p, miR-142-3p, miR-146a-5p, and miR-148b-3p.

In certain embodiments, the stimulating comprises administering an effective amount of sound waves sufficient to increase numbers of extracellular vesicles produced by the mesenchymal stromal cell compared to the numbers of the extracellular vesicles produced by a reference mesenchymal stromal cell that is not stimulated with the sound waves.

In certain embodiments, the mesenchymal stromal cell is from umbilical cord, placental tissue, adipose tissue, or bone marrow.

In certain embodiments, the extracellular vesicles are exosomes, microvesicles, apoptotic bodies, ectosomes, or microparticles.

In certain embodiments, the extracellular vesicles have diameters ranging from about 1 nm to about 2000 nm.

In certain embodiments, the extracellular vesicles comprise one or more surface markers selected from the group consisting of TSG101, ALIX, CD63, and CD9.

In certain embodiments, the mesenchymal stromal cell is adherent or in a suspended population in culture.

In certain embodiments, the mesenchymal stromal cell is a genetically modified mesenchymal stromal cell. In some embodiments, the extracellular vesicles derived from the genetically modified mesenchymal stromal cell after said stimulation with the sound waves comprise a short hairpin RNA (shRNA), a short interfering RNA (siRNA), a microRNA (miRNA), a Clustered Regularly Interspaced Short Palindromic Repeat (CRISPR) guide RNA, or a therapeutic peptide or polypeptide.

In certain embodiments, the method further comprises lyophilizing the extracellular vesicles prior to administering to the subject. In some embodiments, the extracellular vesicles are lyophilized in the presence of a surface-active stabilizer or cryoprotectant.

In certain embodiments, the extracellular vesicles are administered intravenously, intra-arterially, subcutaneously, percutaneously, intramuscularly, intrathecally, by pulmonary inhalation, or locally.

In certain embodiments, the stimulating with sound waves comprises administering pFUS to the mesenchymal stromal cell. For example, pFUS may be administered at an ultrasound frequency ranging from 20 kHz to 3.0 MHz with a pulse repetition frequency (PRF) ranging from 5 Hz to 200 Hz, a ultrasound duty cycle (DC) ranging from 0.1% to 50%, and a peak negative pressure (PNP) ranging from 0.1 MPa to 10 MPa.

In certain embodiments, a single cycle of treatment or multiple cycles of treatment are administered to the subject.

In certain embodiments, the disease associated with cellular-energy deficiency or mitochondrial dysfunction is a lung disease, a kidney disease, or a neurodegenerative disease. In some embodiments, the lung disease is chronic or acute respiratory distress syndrome (ARDS). In some embodiments, the kidney disease is chronic or acute kidney injury (AKI). In some embodiments, the neurodegenerative disease is Alzheimer's disease.

In certain embodiments, the extracellular vesicles are administered with a single route of administration or multiple routes of administration. In some embodiments, the extracellular vesicles derived are administered at a single location or at multiple locations.

In certain embodiments, the method further comprises imaging damaged tissue that is treated with the extracellular vesicles (e.g., before, during, or after treatment). Exemplary medical imaging techniques include, without limitation, ultrasound, magnetic resonance imaging (MRI), computed tomography (CT), or scintigraphy.

In certain embodiments, the method further comprises coculturing the extracellular vesicles derived from the pFUS-stimulated mesenchymal stromal cell with the mesenchymal stromal cell or another type of cell prior to said administering the extracellular vesicles to the subject.

In certain embodiments, the method further comprises administering a cellular therapy to the subject.

In another aspect, a composition is provided, the composition comprising extracellular vesicles derived from a mesenchymal stromal cell that has been stimulated with sound waves for use in a method of treating a disease or condition associated with cellular-energy deficiency or mitochondrial dysfunction.

In certain embodiments, the mesenchymal stromal cell has been stimulated with sound waves by administering pulsed focused ultrasound (pFUS) to the mesenchymal stromal cell. In some embodiments, the pFUS has been administered to the mesenchymal stromal cell at an ultrasound frequency ranging from 20 kHz to 3.0 MHz with a pulse repetition frequency (PRF) ranging from 5 Hz to 200 Hz, a ultrasound duty cycle (DC) ranging from 0.1% to 50%, and a peak negative pressure (PNP) ranging from 0.1 MPa to 10 MPa.

In certain embodiments, the disease associated with cellular-energy deficiency or mitochondrial dysfunction is a lung disease, kidney disease, or a neurodegenerative disease. In some embodiments, the lung disease is chronic or acute respiratory distress syndrome (ARDS). In some embodiments, the kidney disease is chronic or acute kidney injury (AKI). In some embodiments, the neurodegenerative disease is Alzheimer's disease.

In certain embodiments, the composition further comprises a pharmaceutically acceptable excipient.

In another aspect, a method of improving metabolic health of a damaged, exhausted, or diseased cell is provided, the method comprising: stimulating a mesenchymal stromal cell with sound waves; collecting extracellular vesicles secreted from the mesenchymal stromal cell after said stimulating the mesenchymal stromal cell with the sound waves; contacting the damaged, exhausted, or diseased cell with an effective amount of the extracellular vesicles, wherein the metabolic health of the damaged, exhausted, or diseased cell is improved.

In certain embodiments, the contacting is performed in vivo or ex vivo.

In certain embodiments, the method further comprises culturing the damaged, exhausted, or diseased cell in the presence of the extracellular vesicles.

In certain embodiments, the damaged, exhausted, or diseased cell is an immune cell, an epithelial cell, or an endothelial cell. In some embodiments, the immune cell is a macrophage, a dendritic cell, a T cell, a B cell, a natural killer cell, or a monocyte. In some embodiments, the T cell is an exhausted T cell.

In certain embodiments, the method further comprises performing cellular therapy with the damaged, exhausted, or diseased cell after the metabolic health of the damaged, exhausted, or diseased cell is improved, e.g., after said contacting the damaged, exhausted, or diseased cell with the extracellular vesicles. In certain embodiments, the mesenchymal stromal cell has been stimulated with sound waves by administering pulsed focused ultrasound (pFUS) to the mesenchymal stromal cell. In some embodiments, the pFUS has been administered to the mesenchymal stromal cell at an ultrasound frequency ranging from 20 kHz to 3.0 MHz with a pulse repetition frequency (PRF) ranging from 5 Hz to 200 Hz, a ultrasound duty cycle (DC) ranging from 0.1% to 50%, and a peak negative pressure (PNP) ranging from 0.1 MPa to 10 MPa.

### BRIEF DESCRIPTION OF THE DRAWINGS

FIGS. 1A-1F. Pilot data showing UC-MSC perform better than other sources of MSC in reducing inflammation in ARDS in the lungs as indicated by (FIG. 1A) CT, (FIG. 1B) H&E staining (arrow: inflammatory cells; stars: fluid/debris filled alveoli) and (FIG. 1C) overall animal survival. (FIG. 1D) UC-MSCs also inhibit pro-inflammatory M1 macrophage polarization while promoting anti-inflammatory M2 phenotypes. Genomic data shows that UC-MSCs also have the (FIG. 1E) lowest expression of Angiotensin Converting Enzyme (ACE), and (FIG. 1F) higher expression of genes related to for mitochondrial biogenesis, dynamics and structure. In summary, UC-MSCs appear to be the best source of MSCs for the treatment of ARDS.

FIGS. 2A-2F. pFUS stimulation, at low acoustic doses, is safe and can be used to enhance the metabolic health and immunomodulatory properties of cells. FIG. 2A Schematic showing pFUS experimental set up. (FIG. 2B) schematic showing how the mechanical stimulation is provided by soundwaves to UC-MSCs to enhance EV amount and their cargo for maintaining metabolic health of the injured cells. (FIG. 2C)) For optimization of different acoustic dose of pFUS we stimulate UC-MSCs with high, medium and low dose of pFUS where compared to high and medium, dose low dose pFUS showed increase in NADPH dehydrogenase activity, mitochondrial membrane potential, intracellular calcium and decrease in ROS production in UC-MSCs. Moreover, oxidative phosphorylation was significantly increase in low acoustic dose of pFUS suggesting low dose pFUS as the optimized parameter for stimulation of UC-MSCs. (FIG. 2D) All significant (FDR<0.05) pathways related to response to immune response, angiogenesis and cellular metabolic health upregulated in pFUS-UC-MSCs compared to basal conditions, which are relevant in attenuating ARDS. (FIG. 2E) Changes in gene expression related to cellular metabolic health (oxidative phosphorylation and glycolysis and mitochondrial biogenesis), among different UC-MSC donors (n=3), following pFUS. (FIG. 2F) Changes in immunomodulatory and angiogenic secretory profile of UC-MSCs following pFUS. In summary pFUS stimulation, at low acoustic doses, is safe and can be used to enhance metabolic health of the cells and immunomodulatory properties.

FIGS. 3A-3F. Characterization of EVs and EVs cargo. (FIG. 3A) NAT analysis for measurement of concentration of EVs suggesting 1.2-1.3 fold over increase in the number of EVs in pUC-EVs compared to UC-EVs. (FIG. 3B) Western blot showing the expression of CD63 and CD9 expression in the UC-EVs and pUC-EVs. (FIG. 3C) TEM image showing morphology and size of different fraction of EVs (MVs and Exo). (FIG. 3D) NTA analysis showing size and concentration of MVs and Exo. (FIG. 3E) Upper panel showing the mitochondrial inner, outer membrane, and matrix proteins and lower panel showed the western blot for the expression of these proteins in MVs and Exo fraction of UC-EVs and pUC-EVs where we observed increase in mitochondrial protein in the MVs fraction of pUC-EVs compared to UC-EVs and we could not find the expression of these proteins in the exo group both in UC-EVs and pUC-EVs group suggesting the presence of intact mitochondria in the MVs which was upregulated with pFUS. (FIG. 3F) Table showing miRNA that are changed in pUC-EVs and have role in regulating immunomodulation and metabolism.

FIGS. 4A-4D. The pUC-EVs improve the metabolic health of injured A459 lung epithelial cells. A representative confocal microscopy image showing the uptake of MitoTracker Red labelled pFUS-UC-MSC-EVs by lung epithelial (A459) cells following their exposure to an inflammatory cocktail of cytokines: TNF-α+INF-γ at 24 h is shown in FIG. 4A where pUC-EVs treated groups showed the more red signals coming from the mitochondria present the EVs suggesting higher mitochondrial load in pUC_EVs which is being uptaken by the injured cells to regain their bioenergetics health. In addition, oxygen consumption rate (OCR) and extracellular acidification rate (ECAR) in A549 cells showed that there is decreased in oxidative phosphorylation and glycolysis when treated with inflammatory cocktail (i.e Inflammation) which is regained with the pUC-EVs treatment Measurement of lung epithelial cell (FIG. 4B). ATP production using a fluorometric assay kit (Sigma) at 24 h, showed that inflammation decreases cellular ATP production in epithelial cells that can be restored following pFUS-UC-MSC-EV treatment (FIG. 4C). Moreover, apoptosis and necrosis in the A549 cells was reduced in pUC-EVS treated group suggesting the protective role of pUC-EVs in protecting lung epithelial injury (FIG. 4D). In summary, pFUS-UC-MSC-EVs contain functional mitochondria that can help restore ATP synthesis in lung epithelial cells that have been damaged by inflammation.

FIGS. 5A-5C. A representative confocal microscopy image showing the uptake of Mito Tracker Red labelled pFUS-UC-MSC-EVs by macrophages (RAW264.7) following their exposure to an inflammatory cocktail of cytokines: TNF-α+INF-γ at 24 hours is shown in FIG. 5A. Measurement of macrophage polarization, specifically for the M1 phenotype by detecting CD86+ cells, showing this is increased during inflammation but can be significantly reversed when macrophages are exposed to pFUS-UC-MSC-EVs (FIG. 5B). (FIG. 5C) Measurement of macrophage activation to M1 phenotype by detecting CD86 expressing cells where we observed reduction in the CD86+ cells in pUC-EVs treated group compared to UC-EVs and inflammatory cocktail only treated groups. The transfer of functional mitochondria in pUC-EVs helped for metabolic shift of the macrophages to reduce the pro-inflammatory phenotype of macrophages. In summary, pFUS-UC-MSC-EVs contain functional mitochondria that can help reduce the pro-inflammatory M1 phenotype of macrophages.

FIGS. 6A-6D. In vivo effects of (pFUS)-UC-MSC-EVs on immune cells following IV administration. We quantified the number of immune cells (using FACS) present in the BAL of juvenile mice with ARDS following 24 h of LPS administration (FIG. 6A). The data shows increased neutrophil infiltration (CD11b+Ly6G+ cells) and pro-inflammatory M1 polarization of macrophages (M1/M2 ratio), which was not affected by saline or conventional steroid treatment, but which was significantly reduced with pFUS-UC-MSC-EVs delivered IV into mice. FIG. 6B shows that the effect on neutrophils and macrophages in juvenile mice is also dose dependent showing improved therapeutic efficacy at higher doses of pFUS-UC-MSC-EVs. In addition, (FIG. 6C) they have a greater bioenergetic capacity (as indexed by PGC-1α expression) within their lungs at baseline compared to adults, and (FIG. 6D) following inflammation this is completely depleted, but fully is restored with pFUS-UC-MSC-EVs. In summary, pFUS-UC-MSC-EVs offer a very promising clinically translatable therapeutic option to treat ARDS.

FIGS. 7A-7E. Pilot data showing intra-tracheal injection of pUC-EVs perform better than UC-EVs in reducing inflammation in ARDS in the lungs as indicated by (FIG. 7A) CT and H&E staining (*: inflammatory cells infiltration; $: Thickening of alveolar wall), (FIG. 7B) M1/M2 ratio in the BAL where UC-MSCs also inhibit pro-inflammatory M1 macrophage polarization while promoting anti-inflammatory M2 phenotypes (FIG. 7C) % of neutrophil in BAL and (FIG. 7D) neutrophil activity measure by MPO assay where the neutrophil infiltration and activation was significantly lower in the pUC-EVs treated groups. (FIG. 7E) The Normalized counts from lung tissue transcriptome indicating changes in gene expression for genes related to inflammation where the decrease in the inflammation was observed in pUC-EVs treated groups. In summary, pUC-EVs improve the EVs therapy for the treatment of ARDS.

FIGS. 8A-8F. Improvement of bioenergetic health and the viability of neurons with EVs therapy. (FIGS. 8A and 8D) ATP production in neurons after retenone/inflammatory cocktail (TNF α and IFN-γ) treatment where pFUS-UC-MSCs showed improvement in ATP generation compared to UC-MSC-EVs and reduced the cell death suggested by decrease in necrosis (FIGS. 8B and 8E) without change in apoptosis (FIGS. 8C and 8F).

FIGS. 9A-9F. Improvement of bioenergetic health and the viability of microglia with EVs therapy. (FIGS. 9A and 9D) ATP production in neurons after retenone/inflammatory cocktail (TNF α and IFN-γ) treatment where pFUS-UC-MSCs showed improvement in ATP generation and reduced the cell death suggested by decrease in necrosis (FIGS. 9B and 9E) without change in apoptosis (FIGS. 9C and 9F).

FIGS. 10A-10H. Improvement of bioenergetic health and the viability of neurons and microglia with EVs therapy. (FIGS. 10A and 10E) BCL2 expression in neurons and microglia respectively where pFUS-UC-MSCs showed increase in BCL2 expression suggesting reduction in cell death (FIGS. 10B and 10F) NRF2 expression in neurons and microglia respectively suggesting increase in NRF2 expression suggesting the reduction in oxidative stress. (FIGS. 10C and 10F) PGC-1^{α} expression in neurons and microglia respectively, and (FIGS. 10D and 10H) TOM20 expression in neurons and microglia respectively suggesting the mitochondrial biogenesis occurring in neurons and microglia with pFUS-UC-MSC-EVs.

FIG. 11. Effect of pFUS UC-EVs in preventing cisplatin induced AKI. Intra-arterial delivery of pUC-EVs (100 ug/kg) in mouse were able to prevent increase in BUN, sCreatinine. pUC-EVs also increased marker for mitochondrial biogenesis (PGC1A), which may help in restoring bioenergetics in kidney and reduce systemic inflammation (TNFα and IL1B).

### DETAILED DESCRIPTION

Methods for treating diseases and conditions associated with cellular-energy deficiency or mitochondrial dysfunction are provided. The methods utilize extracellular vesicles derived from mesenchymal stromal cells (MSCs) that have been treated with sound waves. The use of pFUS stimulation at low acoustic doses enhances the production and bioenergetic profile of extracellular vesicles from MSCs. The extracellular vesicles derived from MSCs stimulated with sound waves can be used to reduce inflammation, restore the bioenergetic health of injured cells, and promote regeneration of injured tissue through the release of the extracellular vesicle cargo, which contains mitochondria-related products.

Before the treatment methods are further described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As used herein the singular forms “a”, “an”, and “the” include plural referents unless the context clearly dictates otherwise. Thus, for example, reference to “a cell” includes a plurality of such cells, and reference to “the extracellular vesicle” includes reference to one or more extracellular vesicles and equivalents thereof, e.g., exosomes, microvesicles, apoptotic bodies, ectosomes, microparticles, etc., known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “about,” particularly in reference to a given quantity, is meant to encompass deviations of plus or minus five percent.

As used herein, the terms “mesenchymal stromal cells” and “mesenchymal stem cells” are used interchangeably and refer to multipotent cells derived from connective tissue. The terms encompass MSCs derived from various sources including, without limitation, umbilical cord tissue, bone marrow, adipose tissue, molar tooth bud tissue, and amniotic fluid.

As used herein, the term “extracellular vesicle” means a vesicle released by a mammalian cell (e.g., MSC). Examples of “extracellular vesicles” include exosomes, ectosomes, microvesicles, microparticles, and apoptotic bodies.

The term “disease or condition associated with cellular-energy deficiency or mitochondrial dysfunction” is used herein to refer to any disease or condition associated with mitochondrial dysfunction, cellular-energy deficiency, reduced autophagy/mitophagy, or accumulation of damaged mitochondria. Diseases and conditions associated with cellular-energy deficiency or mitochondrial dysfunction include, but are not limited to, mitochondrial diseases such as mitochondrial myopathy, diabetes mellitus and deafness (DAD), Kearns-Sayre, syndrome, Leber's hereditary optic neuropathy (LHON), Leigh syndrome, neuropathy, ataxia, retinitis pigmentosa, and ptosis (NARP), myoneurogenic gastrointestinal encephalopathy (MNGIE), myoclonic epilepsy and ragged red muscle fibers (MERRF) syndrome, mitochondrial myopathy, encephalopathy, lactic acidosis, and stroke-like episodes (MELAS) syndrome, and mitochondrial DNA depletion syndrome; other diseases associated with cellular-energy deficiency or mitochondrial dysfunction, such as lung diseases such as chronic or acute respiratory distress syndrome (ARDS), pneumonia, chronic obstructive pulmonary disease (COPD), bronchial asthma, and idiopathic pulmonary fibrosis (IPF); neurodegenerative disorders including Alzheimer's disease (AD), amyotrophic lateral sclerosis (ALS), Huntington's disease (HD), optic neuropathy (ON), Marie-Charcot-Tooth (MCT) disease, and Parkinson's disease; kidney diseases, including chronic kidney disease (CKD) and acute kidney injury (AKI), mitochondrial tubulopathy, cystic renal disease, and mitochondrial dysfunction-induced kidney injury; liver diseases such as alcoholic fatty liver disease (AFLD) and nonalcoholic fatty liver disease (NAFLD), non-alcoholic steatohepatitis (NASH), alcoholic steatohepatitis (ASH), liver fibrogenesis, cirrhosis, and liver inflammation; heart diseases such as atherosclerosis, ischemia-reperfusion injury, heart failure, cardiac hypertrophy, and hypertension; insulin resistance, diabetes, obesity-associated steatohepatitis, cancer, sarcopenia, chronic fatigue syndrome, low-grade chronic inflammation, and mitochondrial dysfunction associated with ageing, including reduced autophagy/mitophagy and accumulation of damaged mitochondria.

The term “administering” is intended to include routes of administration which allow extracellular vesicles derived from MSCs stimulated with sound waves (e.g., pFUS-stimulated MSC-derived extracellular vesicles) to perform the intended function of reducing inflammation, restoring the bioenergetic health of injured cells, increasing ATP production, and/or promoting regeneration of injured tissue through the release of the extracellular vesicle cargo. Examples of routes of administration which can be used include, but are not limited to, intravenous, pulmonary inhalation (e.g., nebulization for delivery to the lungs), intra-arterial (e.g., for delivery to solid organs such as the kidneys, liver, heart, and brain), intrathecal or direct injection into cerebrospinal fluid (e.g., for delivery to the brain); or intramuscular or percutaneous delivery (e.g., for delivery to muscle). Injections can be administered as bolus injections or by continuous infusion. Depending on the route of administration, extracellular vesicles can be coated with or disposed in a selected material to protect them from natural conditions which may detrimentally affect their ability to perform their intended function. Extracellular vesicles may be administered alone, or in conjunction with a pharmaceutically acceptable carrier. Further, extracellular vesicles may be coadministered with a pharmaceutically acceptable carrier.

By “pFUS-stimulated MSC-derived extracellular vesicles” is meant extracellular vesicles derived from MSCs that are stimulated by administering an effective amount of pFUS sufficient to increase levels in the extracellular vesicles of mitochondrial miRNA, mitochondrial mRNA, mitochondrial proteins, and lipids involved in promoting mitochondrial biogenesis and ATP production compared to their levels in extracellular vesicles from a reference mesenchymal stromal cell that is not stimulated with pFUS.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease.

By “therapeutically effective dose or amount” of extracellular vesicles derived from MSCs stimulated with sound waves (e.g., pFUS-stimulated MSC-derived extracellular vesicles) is intended an amount of the pFUS-stimulated MSC-derived extracellular vesicles that brings about a positive therapeutic response, such as improved recovery from a disease associated with cellular energy deficiency or mitochondrial dysfunction. Improved recovery may include reduced inflammation, improved bioenergetic health of injured or diseased cells, and/or regeneration of injured tissue through the release of the extracellular vesicle cargo. Additionally, a therapeutically effective dose or amount of extracellular vesicles derived from MSCs stimulated with sound waves (e.g., pFUS-stimulated MSC-derived extracellular vesicles) may restore ATP synthesis. A therapeutically effective dose or amount can be administered in one or more administrations.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

“Isolated” refers to an entity of interest that is in an environment different from that in which it may naturally occur. “Isolated” is meant to include entities that are within samples that are substantially enriched for the entity of interest and/or in which the entity of interest is partially or substantially purified.

By “subject” is meant any member of the subphylum Chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

### Administration of Extracellular Vesicles Derived from MSCs Stimulated with Sound Waves

At least one therapeutically effective dose of extracellular vesicles derived from MSCs stimulated with sound waves (e.g., pFUS-stimulated MSC-derived extracellular vesicles) will be administered for treatment of a disease or condition associated with cellular-energy deficiency or mitochondrial dysfunction. Diseases and conditions associated with cellular-energy deficiency or mitochondrial dysfunction include, but are not limited to, mitochondrial diseases such as mitochondrial myopathy, diabetes mellitus and deafness (DAD), Kearns-Sayre, syndrome, Leber's hereditary optic neuropathy (LHON), Leigh syndrome, neuropathy, ataxia, retinitis pigmentosa, and ptosis (NARP), myoneurogenic gastrointestinal encephalopathy (MNGIE), myoclonic epilepsy and ragged red muscle fibers (MERRF) syndrome, mitochondrial myopathy, encephalopathy, lactic acidosis, and stroke-like episodes (MELAS) syndrome, and mitochondrial DNA depletion syndrome; and other diseases associated with cellular-energy deficiency or mitochondrial dysfunction, such as lung diseases, including chronic or acute respiratory distress syndrome (ARDS), pneumonia, chronic obstructive pulmonary disease (COPD), bronchial asthma, and idiopathic pulmonary fibrosis (IPF); neurodegenerative disorders including Alzheimer's disease (AD), amyotrophic lateral sclerosis (ALS), Huntington's disease (HD), optic neuropathy (ON), Marie-Charcot-Tooth (MCT) disease, and Parkinson's disease; kidney diseases, including chronic kidney disease (CKD) and acute kidney injury (AKI), mitochondrial tubulopathy, cystic renal disease, and mitochondrial dysfunction-induced kidney injury; liver diseases such as alcoholic fatty liver disease (AFLD) and nonalcoholic fatty liver disease (NAFLD), non-alcoholic steatohepatitis (NASH), alcoholic steatohepatitis (ASH), liver fibrogenesis, cirrhosis, liver inflammation; heart diseases, including atherosclerosis, ischemia-reperfusion injury, heart failure, cardiac hypertrophy, and hypertension; insulin resistance, diabetes, obesity-associated steatohepatitis, cancer, sarcopenia, chronic fatigue syndrome, low-grade chronic inflammation, and mitochondrial dysfunction associated with ageing, including reduced autophagy/mitophagy and accumulation of damaged mitochondria.

By “therapeutically effective dose or amount” of extracellular vesicles derived from MSCs stimulated with sound waves (e.g., pFUS-stimulated MSC-derived extracellular vesicles) is intended an amount of the extracellular vesicles that brings about a positive therapeutic response, such as improved recovery from a disease associated with cellular energy deficiency or mitochondrial dysfunction. Improved recovery may include reduced inflammation, improved bioenergetic health of injured or diseased cells, and/or regeneration of injured tissue through the release of the extracellular vesicle cargo. Additionally, a therapeutically effective dose or amount of extracellular vesicles derived from MSCs stimulated with sound waves (e.g., pFUS-stimulated MSC-derived extracellular vesicles) may restore ATP synthesis in cells.

The MSCs may be derived from any source including, without limitation, bone marrow, adipose tissue, umbilical cord tissue, placental tissue, molar tooth bud tissue, and amniotic fluid. The MSCs may be obtained directly from the patient to be treated, a donor, a culture of cells from a donor, or from established cell culture lines. In some embodiments, the MSCs are obtained from mammals, including without limitation, humans, non-human primates such as chimpanzees and other apes and monkey species; rodents such as rats, mice, and guinea pigs; and farm animals such as cattle, sheep, pigs, goats and horses. The subject methods may be used for treatment of a human patient, in which case, the MSCs from which the extracellular vesicles are derived, are preferably human. The subject methods may also be useful in veterinarian applications, e.g., for treatment of farm animals or pets, in which case, the MSCs from which the extracellular vesicles are derived, are preferably of the same species as the animal being treated.

In certain embodiments, MSCs are stimulated with an effective amount of sound waves (e.g., pFUS) sufficient to increase levels of mitochondrial miRNA, mitochondrial mRNA, and mitochondrial proteins involved in promoting mitochondrial biogenesis and ATP production in the extracellular vesicles produced by the MSCs. In some embodiments, stimulation of MSCs with sound waves results in production of extracellular vesicles having higher amounts of the COX-IV, Complex I/II, HSP60, citrate synthase, and TOM20 protein, which regulate ATP production. In some embodiments, pFUS stimulation of MSCs results in production of extracellular vesicles having higher amounts of miRNAs such as miR-9-5p, miR-15a-5p, miR-16-5p, miR-18a-5p, miR-19b-3p, miR-20a-5p, miR-29a-3p, miR30a-5p, miR-30b-5p, miR-30e-5p, miR-34a-5p, miR-92a-3p, miR-142-3p, miR-146a-5p, miR-148b-3p, which are involved in regulation of pathways related to mitochondrial biogenesis (PGC-1α), mitochondrial inner membrane formation (MIB complex) and mitochondrial function (NRF1/NRF2, respiratory chain complexes). In some embodiments, MSCs are stimulated with an effective amount of pFUS sufficient to increase numbers of extracellular vesicles produced by the MSCs.

In certain embodiments, the MSCs are stimulated with pFUS with an ultrasound frequency ranging from about 20 kHz to about 3.0 MHz, including any ultrasound frequency within this range, such as 20 kHz, 30 kHz, 40 kHz, 50 kHz, 60 kHz, 70 kHz, 80 kHz, 90 kHz, 100 kHz, 200 kHz, 300 kHz, 400 kHz, 500 kHz, 600 kHz, 700 kHz, 800 kHz, 900 kHz, 1.0 MHz, 1.1 MHz, 1.2 MHz, 1.3 MHz, 1.4 MHz, 1.5 MHz, 1.6 MHz, 1.7 MHz, 1.8 MHz, 1.9 MHz, 2.0 MHz, 2.1 MHz, 2.2 MHz, 2.3 MHz, 2.4 MHz, 2.5 MHz, 2.6 MHz, 2.7 MHz, 2.8 MHz, or 3.0 MHz.

In certain embodiments, the MSCs are stimulated with pFUS with a pulse repetition frequency (PRF) ranging from 5 Hz to 200 Hz, including any PRF with this range, such as 5 Hz, 6 Hz, 7 Hz, 8 Hz, 9 Hz, 10 Hz, 20 Hz, 30 Hz, 40 Hz, 50 Hz, 60 Hz, 70 Hz, 80 Hz, 90 Hz, 100 Hz, 125 Hz, 150 Hz, 175 Hz, 200 Hz.

In certain embodiments, the MSCs are stimulated with pFUS with an ultrasound duty cycle ranging from 0.1% to 50%, including any ultrasound duty cycle within this range such as 0.1%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50%.

In certain embodiments, the MSCs are stimulated with pFUS with a negative peak pressure (NPP) ranging from 0.1 MPa to 10 MPa, including any NPP within this range such as 0.1, 0.2, 0.3, 0.4, 0.5, 0.6, 0.7, 0.8, 0.9, 1, 2, 3, 4, 5, 6, 7, 8, 9, or 10 MPa.

In certain embodiments, the MSCs are stimulated with pFUS with an ultrasound frequency ranging from 20 kHz to 3.0 MHz with a pulse repetition frequency (PRF) ranging from 5 Hz to 200 Hz, a ultrasound duty cycle (DC) ranging from 0.1% to 50%, and a peak negative pressure (PNP) ranging from 0.1 MPa to 10 MPa

In certain embodiments, the MSCs are stimulated with pFUS for a time ranging from about 20 seconds to about 7 minutes, including any amount of time within this range, such as 20 seconds, 25 seconds, 30 seconds, 35 seconds, 40 seconds, 45 seconds, 50 seconds, 55 seconds, 1 minute, 1.25 minutes, 1.5 minutes, 1.75 minutes, 2 minutes, 2.25 minutes, 2.5 minutes, 2.75 minutes, 3 minutes, 3.25 minutes, 3.5 minutes, 3.75 minutes, 4 minutes, 4.25 minutes, 4.5 minutes, 4.75 minutes, 5 minutes, 5.25 minutes,, 5.5 minutes, 5.75 minutes, 6 minutes, 6.25 minutes, 6.5 minutes, 6.75 minutes, or 7 minutes. In some embodiments, the pFUS therapy is administered to the subject for at least 20 seconds. In some embodiments, the MSCs are stimulated with pFUS for a period ranging from about 1 minute to about 5 minutes.

The MSC-derived extracellular vesicles may include, without limitation, exosomes, microvesicles, apoptotic bodies, ectosomes, or other microparticles derived from the plasma membrane of MSCs. In certain embodiments, the MSC-derived extracellular vesicles have diameters ranging from about 1 nm to about 2000 nm, including any diameter within this range such as 1 nm, 2 nm, 3 nm, 4 nm, 5 nm, 6 nm, 7 nm, 8 nm, 9 nm, 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm, 1200 nm, 1400 nm, 1600 nm, 1800 nm, or 2000 nm. In certain embodiments, the MSC-derived extracellular vesicles have diameters ranging from about 20 nm to about 180 nm.

In certain embodiments, the MSC, from which the extracellular vesicles are derived, is genetically modified. MSCs may be genetically modified to express an agent such as, but not limited to, a short hairpin RNA (shRNA), a short interfering RNA (siRNA), a microRNA (miRNA), a Clustered Regularly Interspaced Short Palindromic Repeat (CRISPR) guide RNA, or a therapeutic peptide, polypeptide, or protein, wherein the genetically modified MSC secretes the agent in extracellular vesicles. MSCs may be genetically modified, for example, by viral mediated gene transfer using viral vectors such as, but not limited to, lentivirus, adenovirus, retroviruses, adeno-associated virus, or herpes virus vectors. See, e.g., Warnock et al. (2011) Methods Mol. Biol. 737:1-25; Walther et al. (2000) Drugs 60(2):249-271; and Lundstrom (2003) Trends Biotechnol. 21(3):117-122; herein incorporated by reference. Alternatively, the genome of a MSC can be modified using engineered nucleases such as, but not limited to, CRISPR/CAS9, meganucleases, zinc finger nucleases (ZFNs), or transcription activator-like effector nucleases (TALENs) for gene editing. See, e.g., CRISPR Gene Editing: Methods and Protocols (edited by Luo, Humana, 2019), Genome Editing and Engineering: From TALENs, ZFNs and CRISPRs to Molecular Surgery (edited by Appasani and Church, Cambridge University Press, 2018); herein incorporated by reference in their entireties.

The extracellular vesicles derived from MSCs stimulated with sound waves (e.g., pFUS-stimulated MSC-derived extracellular vesicles) may be administered in accordance with any medically acceptable method known in the art. Examples of routes of administration which can be used include, but are not limited to, intravenous, pulmonary inhalation (e.g., nebulization for delivery to the lungs), intra-arterial (e.g., for delivery to solid organs such as the kidneys, liver, heart, and brain), intrathecal or direct injection into cerebrospinal fluid (e.g., for delivery to the brain); or intramuscular or percutaneous delivery (e.g., for delivery to muscle). Injections can be administered as bolus injections or by continuous infusion. In some embodiments, extracellular vesicles are admininistered using a single route of administration. In other embodiments, extracellular vesicles are admininistered using multiple routes of administration. Extracellular vesicles may be administered at a single site or at multiple sites.

In certain embodiments, multiple therapeutically effective doses of the extracellular vesicles derived from MSCs stimulated with sound waves (e.g., pFUS-stimulated MSC-derived extracellular vesicles) will be administered to the subject. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, and so forth. For example, in some embodiments, the extracellular vesicles will be administered twice-weekly or thrice-weekly for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of the extracellular vesicles are administered to the subject within a 7-day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7-day period, allowing for a minimum of 48 hours between doses and a maximum of 72 hours between doses. For purposes of the present disclosure, this type of dosing is referred to as “intermittent” therapy. In accordance with the methods of the present invention, a subject can receive intermittent therapy (i.e., twice-weekly or thrice-weekly administration of a therapeutically effective dose) for one or more weekly cycles until the desired therapeutic response is achieved. In some embodiments, the therapy is administered for at least 1 week, at least 2 weeks, at least 3 weeks, or at least 4 weeks or longer.

In some embodiments, extracellular vesicles derived from MSCs stimulated with sound waves (e.g., pFUS-stimulated MSC-derived extracellular vesicles) are administered to a subject in combination with a cellular therapy for treatment of a disease or condition associated with cellular-energy deficiency or mitochondrial dysfunction. For example, cellular therapies may include, without limitation, delivery of stem cells such as human embryonic stem cells, neural stem cells, mesenchymal stem cells, or hematopoietic stem cells, progenitor cells, cells that secrete cytokines, chemokines, growth factors, or hormones, differentiated or mature cells, or genetically modified cells. The extracellular vesicles derived from MSCs stimulated with sound waves (e.g., pFUS-stimulated MSC-derived extracellular vesicles) can be administered prior to, concurrent with, or subsequent to the cellular therapy. If provided at the same time as the cellular therapy, the extracellular vesicles can be provided in the same or in a different composition than that used to deliver the cells used in the cellular therapy. Thus, the extracellular vesicles and cells can be presented to the individual by way of concurrent therapy. By “concurrent therapy” is intended administration to a subject such that the therapeutic effect of the combination of the extracellular vesicles and the cellular therapy is caused in the subject undergoing treatment. For example, concurrent therapy may be achieved by administering at least one therapeutically effective dose of a pharmaceutical composition comprising extracellular vesicles derived from MSCs stimulated with sound waves (e.g., pFUS-stimulated MSC-derived extracellular vesicles) and at least one therapeutically effective dose of a pharmaceutical composition comprising the cells used in the cellular therapy according to a particular dosing regimen. Administration of the separate pharmaceutical compositions can be at the same time (i.e., simultaneously) or at different times (i.e., sequentially, in either order, on the same day, or on different days), so long as the therapeutic effect of the combination is caused in the subject undergoing therapy. In certain embodiments, multiple therapeutically effective doses of the extracellular vesicles derived from MSCs stimulated with sound waves (e.g., pFUS-stimulated MSC-derived extracellular vesicles) and the cellular therapy are administered to the subject.

### Isolation of MSC-Derived Extracellular Vesicles

MSCs can be cultured in any suitable media followed by isolation of extracellular vesicles secreted into the media after stimulation of the MSCs with sound waves (e.g., pFUS). Suitable protocols for culturing MSCs and isolating extracellular vesicles are known in the art (see, e.g., Kim et al. (2016) Proc. Natl. Acad. Sci. U.S.A., 113(1):170-175; herein incorporated by reference in its entirety). For example, MSCs can be cultured in media containing 20% fetal bovine serum (FBS), 100 U/mL penicillin and streptomycin (Thermo Fisher Scientific, USA), at room temperature with 5% CO₂ until passage 3. After passage 3, MSCs can be cultured in serum-free Dulbecco's Modified Eagle's Medium (DMEM) until 80%-90% confluency at room temperature. The MSCs may be in an adherent or in a suspended population in culture. In some embodiments, the extracellular vesicles derived from MSCs stimulated with sound waves (e.g., pFUS-stimulated MSC-derived extracellular vesicles) may be cultured with the mesenchymal stromal cells or another type of cell prior to administering the extracellular vesicles to a subject.

To isolate extracellular vesicles from a culture, first cellular debris may be removed from the media by centrifugation (e.g., 5,000×g for 10 minutes at room temperature). The extracellular vesicles can then be isolated from the supernatant, for example, by ultracentrifugation (e.g., at 17,000×g for 20 minutes).

Different types of extracellular vesicles can be distinguished by their surface markers and size. Exosomes typically range in size from 30 nm to 100 nm, whereas microvesicles typically range in size from 0.1 μm to 1.0 μm. The size and number of extracellular vesicles can be determined by various techniques known in the art including, without limitation, transmission electron microscopy (TEM), atomic force microscopy, nanoparticle tracking analysis, flow cytometry, and dynamic light scattering.

Samples can be enriched for extracellular vesicles with particular surface markers by positive selection, negative selection, or a combination thereof. For example, samples can be enriched using capture agents (e.g., antibodies or aptamers that bind selectively to cellular markers on extracellular vesicles) conjugated to magnetic or paramagnetic beads by magnetic separation techniques or by flow cytometry or other sorting methods. In some embodiments, surface markers, which are found on a target extracellular vesicle, are used for positive enrichment of a target extracellular vesicle. In other embodiments, cell surface markers, which are not found on the target extracellular vesicle, are used for negative enrichment by depleting the vesicle population of non-target vesicles.

After isolation, extracellular vesicles may be suspended in a carrier, diluent, vehicle, excipient, or the like suitable for administration, which may include a salt, buffer, antioxidant (e.g., ascorbic acid and sodium bisulfate), preservative (e.g., benzyl alcohol, methyl parabens, ethyl or n-propyl, p-hydroxybenzoate), emulsifying agent, suspending agent, dispersing agent, solvent, filler, bulking agent, detergent, and/or adjuvant. For example, a suitable vehicle may include physiological saline solution or buffered saline, supplemented with other materials common in pharmaceutical compositions, e.g., for parenteral administration. Neutral buffered saline or saline mixed with serum albumin are further exemplary vehicles. Those skilled in the art will readily recognize a variety of buffers that could be used in the compositions containing extracellular vesicles. Buffers include, but are not limited to, pharmaceutically acceptable weak acids, weak bases, or mixtures thereof. For example, the buffer components can be water soluble materials such as phosphoric acid, tartaric acids, lactic acid, succinic acid, citric acid, acetic acid, ascorbic acid, aspartic acid, glutamic acid, and salts thereof. Exemplary buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

The extracellular vesicles derived from mesenchymal stromal cells may be stored, e.g., at −80° C. prior to use. In some embodiments, the extracellular vesicles are lyophilized, e.g., in the presence of a surface-active stabilizer or cryoprotectant. Exemplary cryoprotectants include, without limitation, sucrose, trehalose, ethylene glycol, propylene glycol, 2-methyl-2,4-pentanediol, and glycerol. For a description of various surface-active stabilizers, cryoprotectants, and methods of lyophilizing extracellular vesicles, see, e.g., Trenkenschuh et al. (2022) Adv. Healthc. Mater. 11(5):e2100538, Charoenviriyakul et al. (2018) Int. J. Pharm. 553(1-2):1-7, Yuan et al. (2021) Drug Deliv. 28(1):1501-1509, Bahr et al. (2020) Int. J. Vet. Sci. Med. ; 8(1):1-8, Guarro et al. (2022) Colloids Surf. B Biointerfaces 218:112745; herein incorporated by reference.

### Improving Metabolic Health of a Damaged, Exhausted, or Diseased Cell

In certain embodiments, the extracellular vesicles derived from MSCs stimulated with sound waves (e.g., pFUS-stimulated MSC-derived extracellular vesicles) are used to improve the metabolic health of a damaged, exhausted, or diseased cell. For example, damaged, exhausted, or diseased cells may be contacted with an effective amount of the extracellular vesicles in vivo or ex vivo, wherein the metabolic health of the damaged, exhausted, or diseased cell is improved. In certain embodiments, the method further comprises culturing the damaged, exhausted, or diseased cell in a suitable media in the presence of the extracellular vesicles.

The damaged, exhausted, or diseased cell may be any type of cell that would benefit from the extracellular vesicle cargo. In certain embodiments, the damaged, exhausted, or diseased cell is an immune cell, an epithelial cell, or an endothelial cell. In some embodiments, the immune cell is a macrophage, a dendritic cell, a T cell, a B cell, a natural killer cell, or a monocyte. In some embodiments, the T cell is an exhausted T cell.

In certain embodiments, the method further comprises performing cellular therapy with the cell after the metabolic health of the cell is improved from contacting the cell with the extracellular vesicles. For example, damaged, exhausted, or diseased cells may be obtained directly from the patient to be treated, restored by treatment with an effective amount of extracellular vesicles derived from MSCs stimulated with sound waves (e.g., pFUS-stimulated MSC-derived extracellular vesicles) ex vivo, and reimplanted in the patient.

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure numbered 1-46 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A method of treating a subject for a disease or condition associated with cellular-energy deficiency or mitochondrial dysfunction, the method comprising:
stimulating a mesenchymal stromal cell with sound waves; and
administering to the subject a therapeutically effective amount of extracellular vesicles derived from the mesenchymal stromal cell after said stimulating the mesenchymal stromal cell with the sound waves.
2. The method of aspect 1, wherein said stimulating comprises administering an effective amount of the sound waves sufficient to increase levels in the extracellular vesicles of a mitochondrial microRNA (miRNA), a mitochondrial messenger RNA (mRNA), a mitochondrial protein, lipids, or a combination thereof, compared to the levels in extracellular vesicles produced by a reference mesenchymal stromal cell that is not stimulated with the sound waves.
3. The method of aspect 2, wherein the mitochondrial miRNA, mRNA, or protein is involved in promoting mitochondrial biogenesis or production of adenosine triphosphate (ATP).
4. The method of aspect 2 or 3, wherein the mitochondrial protein is COX-IV, TOM20, Complex I, Complex II, Complex II and Complex IV, citrate synthase, HSP60, PGC-1α, SIRT1, SIRT2, SIRT3, MFN, OPA1, DRP1, TRPC, PMCA, RhoA1, Miro1, or mtHSP70.
5. The method of any one of aspects 2-4, wherein the mitochondrial miRNA regulates immunomodulation or metabolic health.
6. The method of aspect 5, wherein the miRNA regulating immunomodulation is mir-9-5p, miR-15a-5p, miR-22-3p, miR-224-3p, miR-144-3p, or miR-146a-5p.
7. The method of aspect 5, wherein the miRNA regulating metabolic health is miR-9-5p, miR-15a-5p, miR-16-5p, miR-18a-5p, miR-19b-3p, miR-20a-5p, miR-29a-3p, miR30a-5p, miR-30b-5p, miR-30e-5p, miR-34a-5p, miR-92a-3p, miR-142-3p, miR-146a-5p, or miR-148b-3p.
8. The method of any one of aspects 1-7, wherein said stimulating comprises administering an effective amount of the sound waves sufficient to increase numbers of extracellular vesicles produced by the mesenchymal stromal cell compared to the numbers of the extracellular vesicles produced by a reference mesenchymal stromal cell that is not stimulated with the sound waves.
9. The method of any one of aspects 1-8, wherein the mesenchymal stromal cell is from umbilical cord, placental tissue, adipose tissue, or bone marrow.
10. The method of any one of aspects 1-9, wherein the extracellular vesicles are exosomes, microvesicles, apoptotic bodies, ectosomes, or microparticles.
11. The method of any one of aspects 1-10, wherein the extracellular vesicles have diameters ranging from about 1 nm to 2000 nm.
12. The method of any one of aspects 1-11, wherein the extracellular vesicles comprise one or more surface markers selected from the group consisting of TSG101, ALIX, CD63, and CD9.

23
13. The method of any one of aspects 1-12, wherein the mesenchymal stromal cell is adherent or in a suspended population in culture.
14. The method of any one of aspects 1-13, wherein the mesenchymal stromal cell is a genetically modified mesenchymal stromal cell.
15. The method of aspect 14, wherein the extracellular vesicles derived from the genetically modified mesenchymal stromal cell after said stimulation with the sound waves comprise a short hairpin RNA (shRNA), a short interfering RNA (siRNA), a microRNA (miRNA), a Clustered Regularly Interspaced Short Palindromic Repeat (CRISPR) guide RNA, or a therapeutic peptide, polypeptide, or protein.
16. The method of any one of aspects 1-15, further comprising lyophilizing the extracellular vesicles prior to administering to the subject.
17. The method of aspect 16, wherein the extracellular vesicles are lyophilized in the presence of a surface-active stabilizer or cryoprotectant.
18. The method of any one of aspects 1-17, wherein the extracellular vesicles are administered intravenously, intra-arterially, subcutaneously, percutaneously, intramuscularly, intrathecally, by pulmonary inhalation, or locally.
19. The method of any one of aspects 1-18, wherein said stimulating comprises administering pulsed focused ultrasound (pFUS) to the mesenchymal stromal cell.
20. The method of aspect 19, wherein the pFUS is administered to the mesenchymal stromal cell at an ultrasound frequency ranging from 20 kHz to 3.0 MHz with a pulse repetition frequency (PRF) ranging from 5 Hz to 200 Hz, a ultrasound duty cycle (DC) ranging from 0.1% to 50%, and a peak negative pressure (PNP) ranging from 0.1 MPa to 10 MPa.
21. The method of any one of aspects 1-20, wherein a single cycle of treatment or multiple cycles of treatment are administered to the subject.
22. The method of any one of aspects 1-21, wherein the disease or condition associated with cellular-energy deficiency or mitochondrial dysfunction is a mitochondrial disease, an inflammatory disease, a hereditary disease, an infection, a degenerative disease, a cardiovascular disease, aging, infarction, chronic fatigue syndrome, or cancer.
23. The method of any one of aspects 1-22, wherein the disease associated with cellular-energy deficiency or mitochondrial dysfunction is a lung disease, kidney disease, or a neurodegenerative disease.
24. The method of aspect 23, wherein the lung disease is chronic or acute respiratory distress syndrome (ARDS).
25. The method of aspect 23, wherein the kidney disease is chronic or acute kidney injury (AKI).
26. The method of aspect 23, wherein the neurodegenerative disease is Alzheimer's disease.
27. The method of any one of aspects 1-26, wherein the extracellular vesicles are administered with a single route of administration or multiple routes of administration.
28. The method of any one of aspects 1-27, further comprising imaging damaged tissue before, during, or after said administering the extracellular vesicles.
29. The method of aspect 28, wherein said imaging is performed by ultrasound, magnetic resonance imaging (MRI), computed tomography (CT), or scintigraphy.
30. The method of any one of aspects 1-29, further comprising coculturing the extracellular vesicles with the mesenchymal stromal cell or another type of cell prior to said administering the extracellular vesicles to the subject.
31. The method of any one of aspects 1-30, further comprising administering a cellular therapy to the subject.
32. A composition comprising extracellular vesicles derived from a mesenchymal stromal cell that has been stimulated with sound waves for use in a method of treating a disease or condition associated with cellular-energy deficiency or mitochondrial dysfunction.
33. The composition of aspect 32, wherein the mesenchymal stromal cell has been stimulated with sound waves by administering pulsed focused ultrasound (pFUS) to the mesenchymal stromal cell.
34. The composition of aspect 33, wherein the pFUS has been administered to the mesenchymal stromal cell at an ultrasound frequency ranging from 20 kHz to 3.0 MHz with a pulse repetition frequency (PRF) ranging from 5 Hz to 200 Hz, a ultrasound duty cycle (DC) ranging from 0.1% to 50%, and a peak negative pressure (PNP) ranging from 0.1 MPa to 10 MPa.
35. The composition of any one of aspects 32-34, wherein the disease associated with cellular-energy deficiency or mitochondrial dysfunction is a lung disease, kidney disease, or a neurodegenerative disease.
36. The composition of aspect 35, wherein the lung disease is chronic or acute respiratory distress syndrome (ARDS).
37. The composition of aspect 35, wherein the kidney disease is chronic or acute kidney injury (AKI).
38. The composition of aspect 35, wherein the neurodegenerative disease is Alzheimer's disease.
39. The composition of any one of aspects 32-38, further comprising a pharmaceutically acceptable excipient.
40. A method of improving metabolic health of a damaged, exhausted, or diseased cell, the method comprising:
stimulating a mesenchymal stromal cell with sound waves;
collecting extracellular vesicles secreted from the mesenchymal stromal cell after said stimulating the mesenchymal stromal cell with the sound waves;
contacting the damaged, exhausted, or diseased cell with an effective amount of the extracellular vesicles, wherein the metabolic health of the damaged, exhausted, or diseased cell is improved.
41. The method of aspect 40, wherein said contacting is performed in vivo or ex vivo.
42. The method of aspect 40, further comprising culturing the damaged, exhausted, or diseased cell in the presence of the extracellular vesicles.
43. The method of any one of aspects 40-42, wherein the damaged, exhausted, or diseased cell is an immune cell, an epithelial cell, or an endothelial cell.
44. The method of aspect 43, wherein the immune cell is a macrophage, a dendritic cell, a T cell, a B cell, a natural killer cell, or a monocyte.
45. the Method of Aspect 44, Wherein the T Cell Is an Exhausted T Cell.
46. The method of any one of aspects 40-45, further comprising performing cellular therapy with the damaged, exhausted, or diseased cell after the metabolic health of the damaged, exhausted, or diseased cell is improved from said contacting the damaged, exhausted, or diseased cell with the extracellular vesicles.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Treating Lung, Kidney, and Neurodegenerative Diseases by Locoregional Delivery of Extracellular Vesicles that Have a Cargo with an Enhanced Bioenergetic Profile

### Introduction

Mitochondrial dysfunction plays a critical role in the initiation and progression of a variety of human diseases, including chronic or acute respiratory distress syndrome (ARDS), pneumonia, chronic obstructive pulmonary disease (COPD), bronchial asthma, idiopathic pulmonary fibrosis (IPF), neurodegenerative diseases, and acute kidney injury. Aging also affects the physiology of the lungs, including reduced autophagy/mitophagy, accumulation of damaged mitochondria, and low-grade chronic inflammation. Here, we created a novel cell-free therapy in the form of umbilical cord mesenchymal stem cell-derived extracellular vesicles (UC-MSC-EVs), which we reliably produced with an enhanced bioenergetic cargo using soundwaves generated by a novel technology called pulsed focused ultrasound (pFUS).

### Results

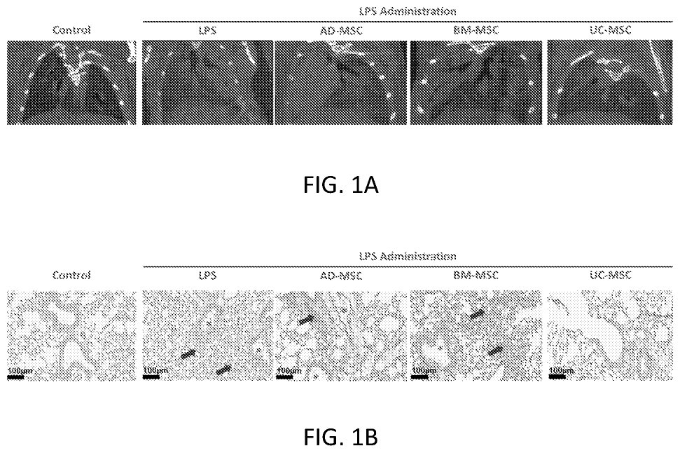

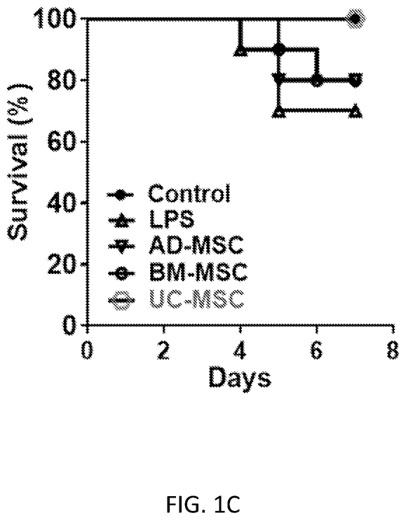

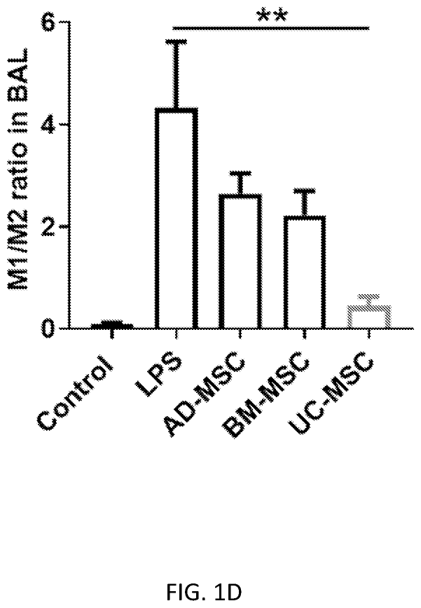

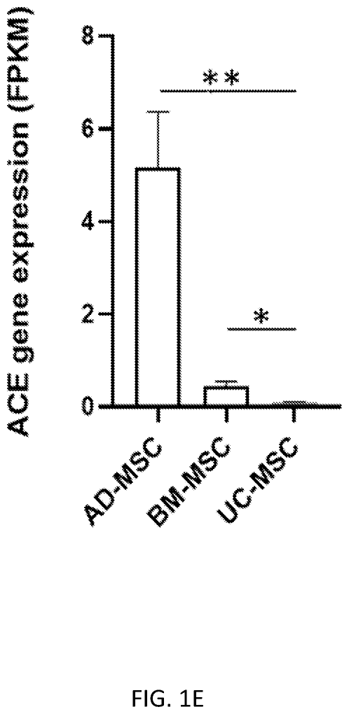

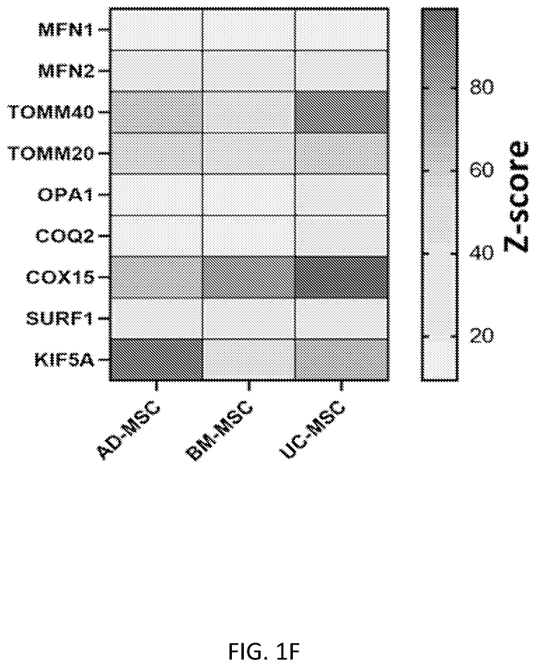

UC-MSC performed better than other sources of MSC in reducing inflammation in ARDS in the lungs as indicated by (FIG. 1A) CT, (FIG. 1B) H&E staining (arrow: inflammatory cells; stars: fluid/debris filled alveoli) and (FIG. 1C) overall animal survival. As shown in FIG. 1D, UC-MSCs also inhibit pro-inflammatory M1 macrophage polarization while promoting anti-inflammatory M2 phenotypes. Genomic data shows that UC-MSCs also have the (FIG. 1E) lowest expression of Angiotensin Converting Enzyme (ACE), and (FIG. 1F) higher expression of genes related to for mitochondrial biogenesis, dynamics and structure. In summary, UC-MSCs appear to be the best source of MSCs for the treatment of ARDS.

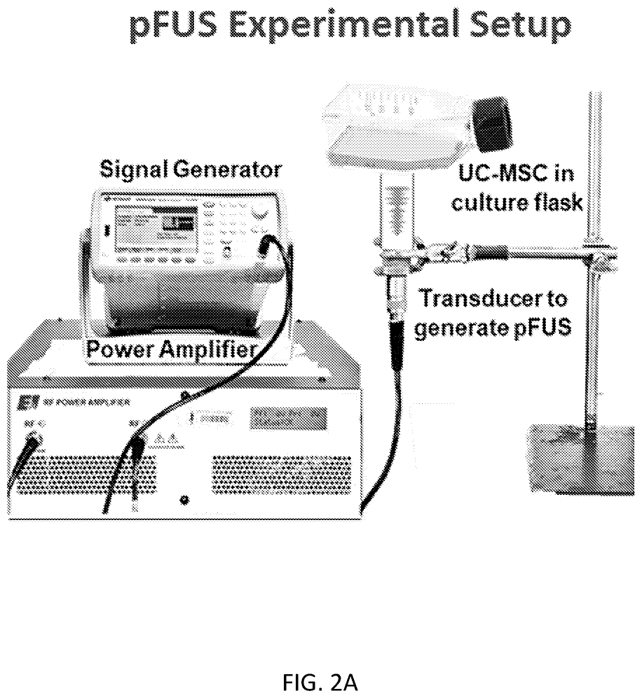

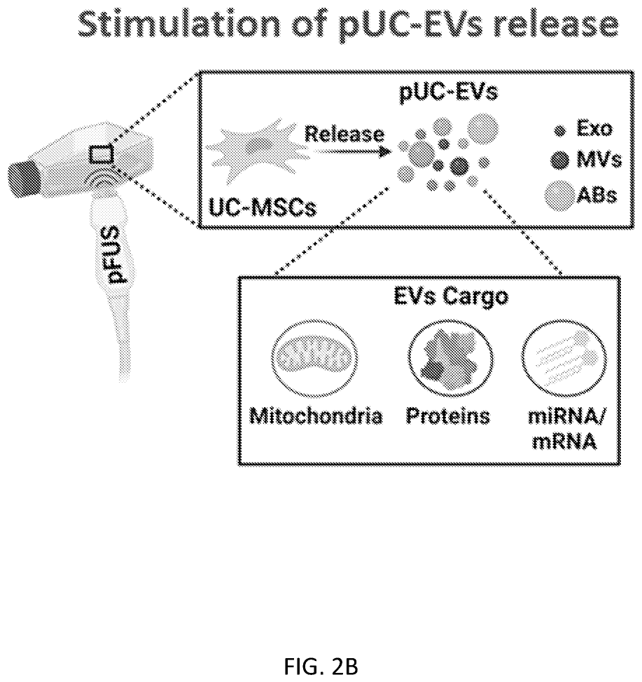

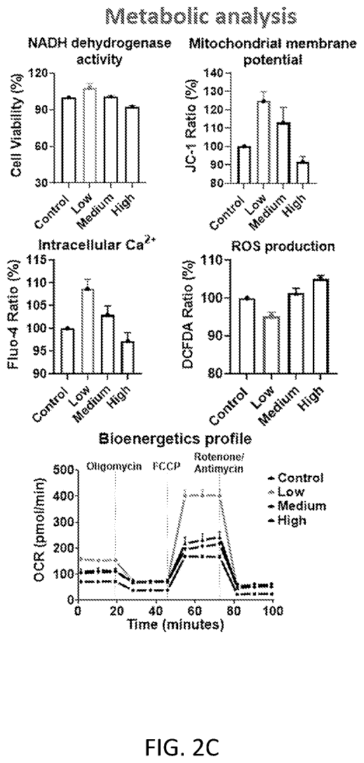

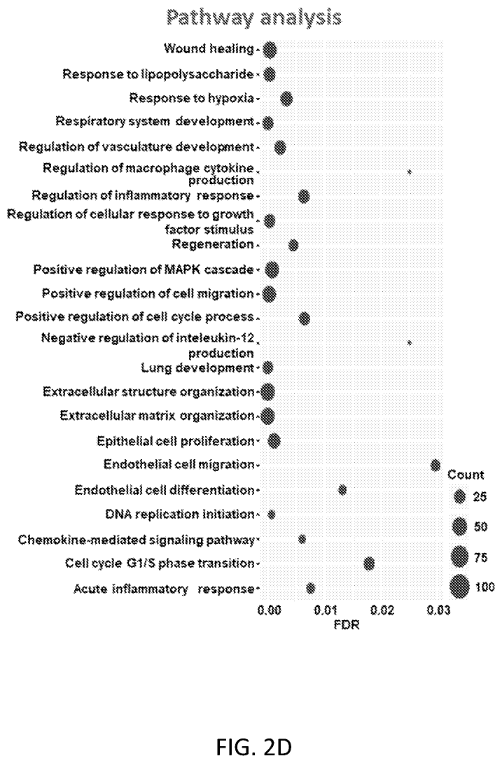

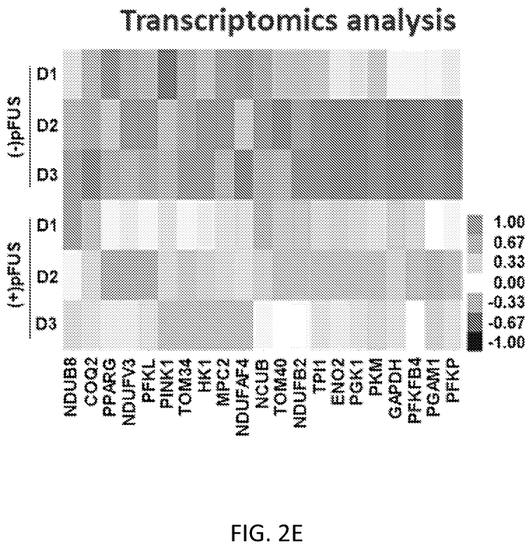

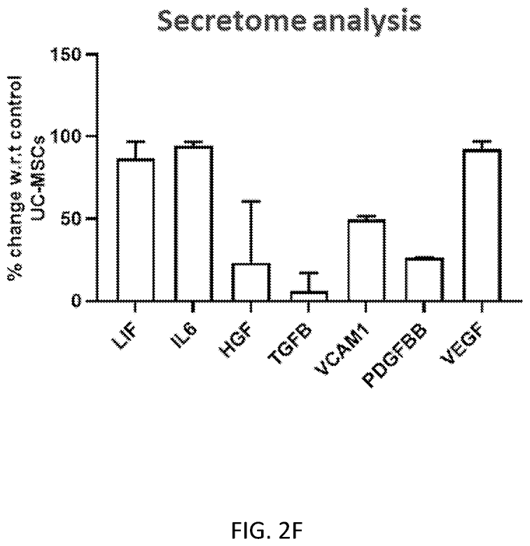

FIG. 2A shows a schematic showing the pFUS experimental set up. FIG. 2B shows a schematic showing how the mechanical stimulation is provided by soundwaves to UC-MSCs to enhance EV amount and their cargo for maintaining metabolic health of the injured cells. For optimization of different acoustic doses of pFUS, we stimulated UC-MSCs with high, medium, and low doses of pFUS for comparison. Low doses of pFUS showed an increase in NADPH dehydrogenase activity, mitochondrial membrane potential, intracellular calcium and decrease in ROS production in UC-MSCs (FIG. 2C). Moreover, oxidative phosphorylation was significantly increased in low acoustic dose of pFUS suggesting low dose pFUS as the optimized parameter for stimulation of UC-MSCs. (FIG. 2D) All significant (FDR<0.05) pathways related to response to immune response, angiogenesis and cellular metabolic health upregulated in pFUS-UC-MSCs compared to basal conditions, which are relevant in attenuating ARDS. FIG. 2E shows changes in gene expression related to cellular metabolic health (oxidative phosphorylation and glycolysis and mitochondrial biogenesis), among different UC-MSC donors (n=3), following pFUS. FIG. 2F shows changes in immunomodulatory and angiogenic secretory profile of UC-MSCs following pFUS. In summary pFUS stimulation, at low acoustic doses, is safe and can be used to enhance metabolic health of the cells and immunomodulatory properties.

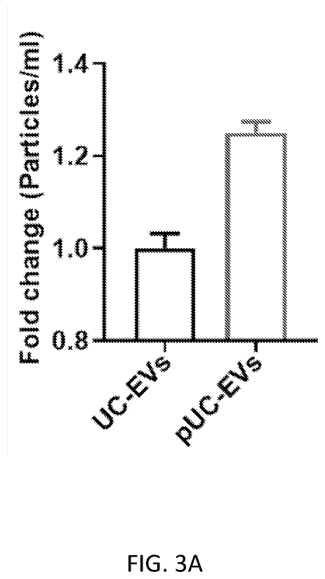

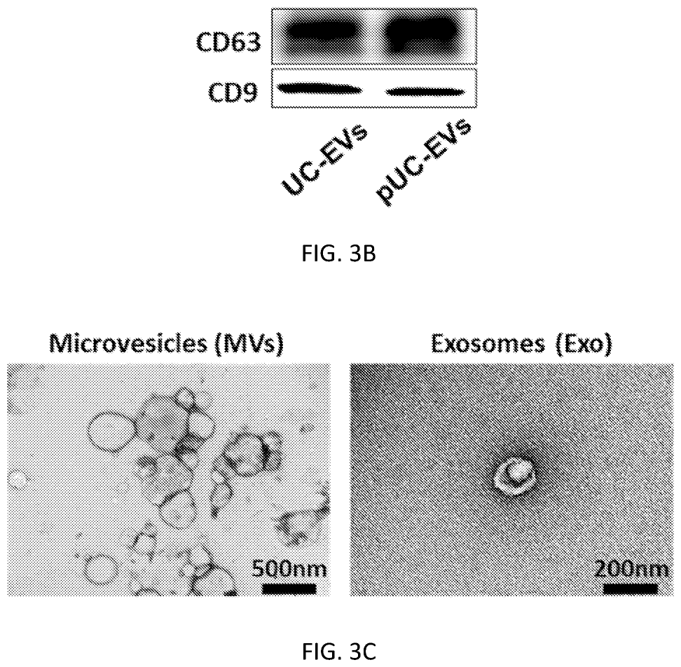

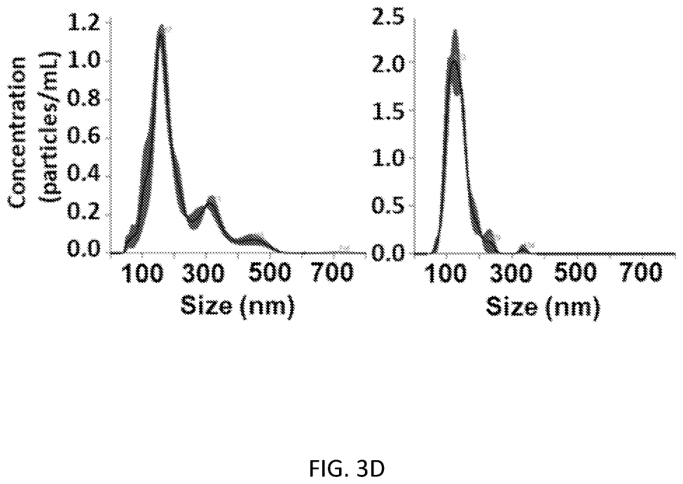

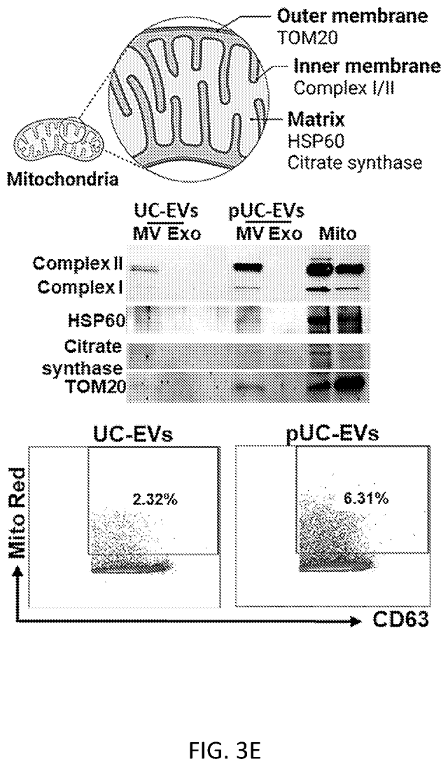

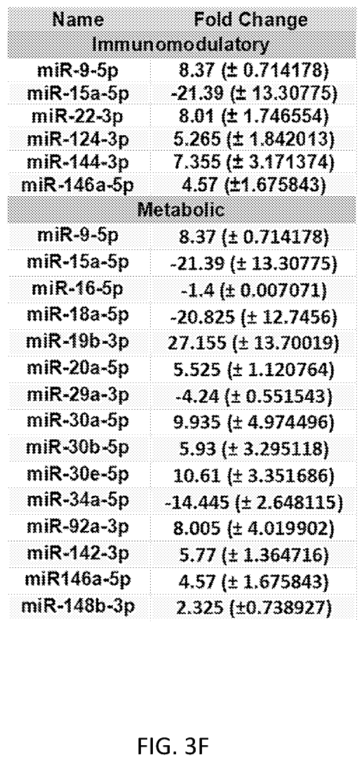

Characterization of EVs and EVs cargo. FIG. 3A shows NAT analysis for measurement of concentration of EVs suggesting 1.2-1.3 fold over increase in the number of EVs in pUC-EVs compared to UC-EVs. FIG. 3B shows a Western blot showing the expression of CD63 and CD9 expression in the UC-EVs and pUC-EVs. FIG. 3C shows a TEM image showing morphology and size of different fraction of EVs (MVs and Exo). FIG. 3D shows a NTA analysis showing size and concentration of MVs and Exo. FIG. 3E (upper panel) shows the mitochondrial inner, outer membrane, and matrix proteins. FIG. 3E (lower panel) shows a western blot for the expression of these proteins in MVs and Exo fraction of UC-EVs and pUC-EVs where we observed an increase in mitochondrial proteins in the MVs fraction of pUC-EVs compared to UC-EVs, and we could not find the expression of these proteins in the exo group both in UC-EVs and pUC-EVs group suggesting the presence of intact mitochondria in the MVs which was upregulated with pFUS. FIG. 3F shows a table showing miRNA that are changed in pUC-EVs and have roles in regulating immunomodulation and metabolism.

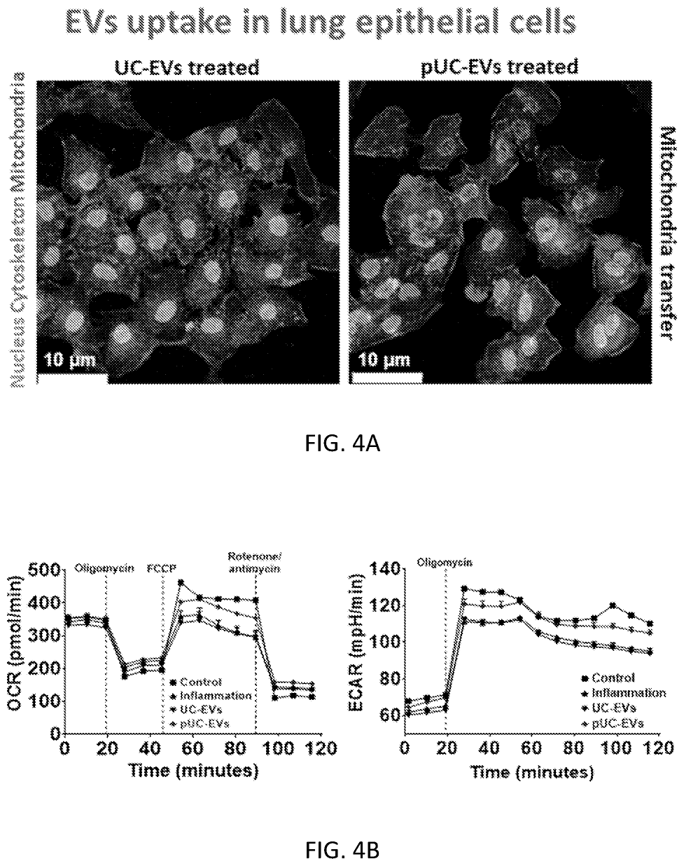

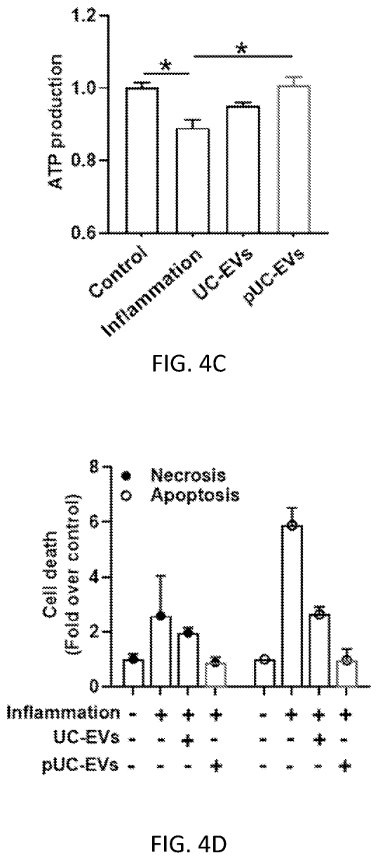

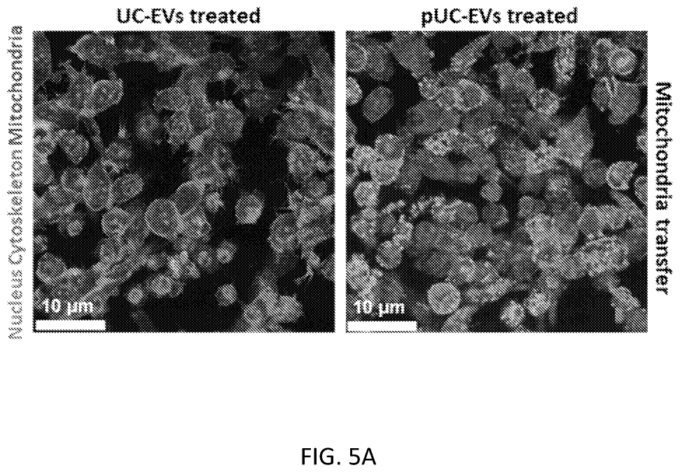

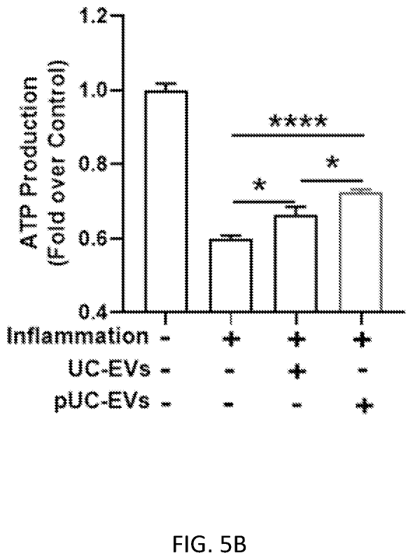

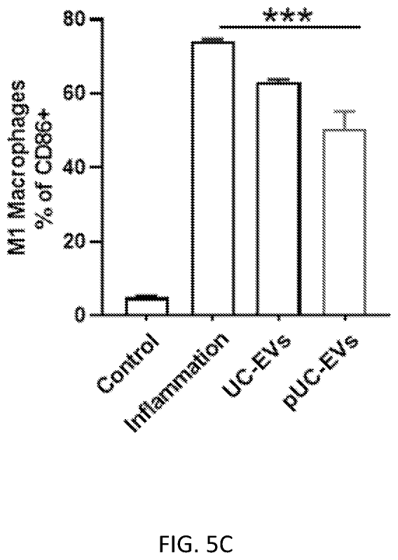

A representative confocal microscopy image showing the uptake of MitoTracker Red labelled pFUS-UC-MSC-EVs by lung epithelial (A459) cells following their exposure to an inflammatory cocktail of cytokines: TNF-α+INF-γ at 24 h is shown in FIG. 4A where pUC-EVs treated groups showed the more red signals coming from the mitochondria present the EVs suggesting higher mitochondrial load in pUC_EVs which is being uptaken by the injured cells to regain their bioenergetics health. In addition, oxygen consumption rate (OCR) and extracellular acidification rate (ECAR) in A549 cells showed that there is decreased in oxidative phosphorylation and glycolysis when treated with inflammatory cocktail (i. e Inflammation) which is regained with the pUC-EVs treatment Measurement of lung epithelial cell (FIG. 4B). ATP production using a fluorometric assay kit (Sigma) at 24 h, showed that inflammation decreases cellular ATP production in epithelial cells that can be restored following pFUS-UC-MSC-EV treatment (FIG. 4C). Moreover, apoptosis and necrosis in the A549 cells was reduced in pUC-EVS treated group suggesting the protective role of pUC-EVs in protecting lung epithelial injury (FIG. 5D) In summary, pFUS-UC-MSC-EVs contain functional mitochondria that can help restore ATP synthesis in lung epithelial cells that have been damaged by inflammation.

A representative confocal microscopy image showing the uptake of MitoTracker Red labelled pFUS-UC-MSC-EVs by macrophages (RAW264.7) following their exposure to an inflammatory cocktail of cytokines: TNF-α+INF-γ at 24 h is shown in FIG. 5A. Measurement of macrophage polarization, specifically for the M1 phenotype by detecting CD86+ cells, showing this is increased during inflammation but can be significantly reversed when macrophages are exposed to pFUS-UC-MSC-EVs (FIG. 5B). In summary, pFUS-UC-MSC-EVs contain functional mitochondria that can help reduce the pro-inflammatory M1 phenotype of macrophages.

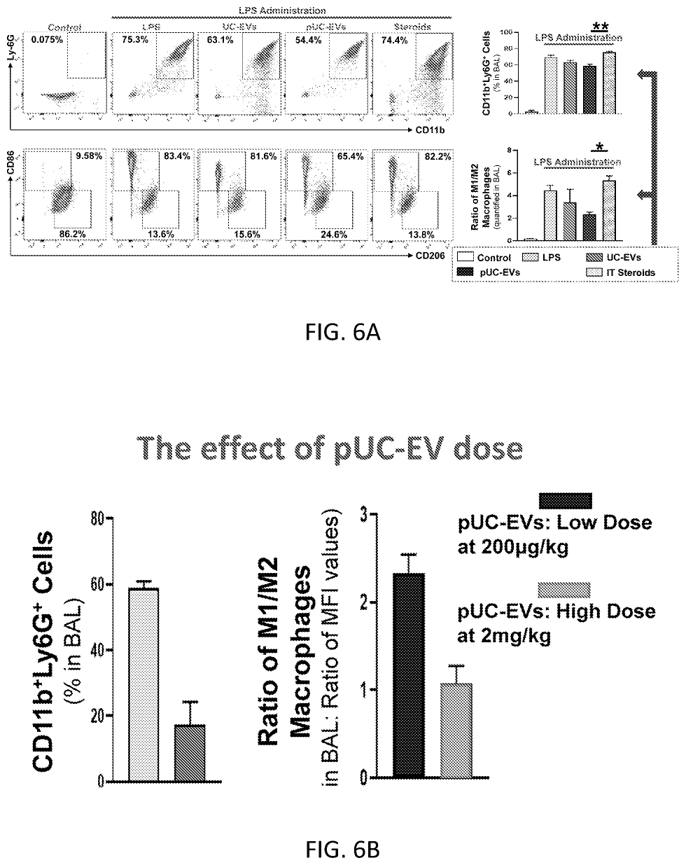

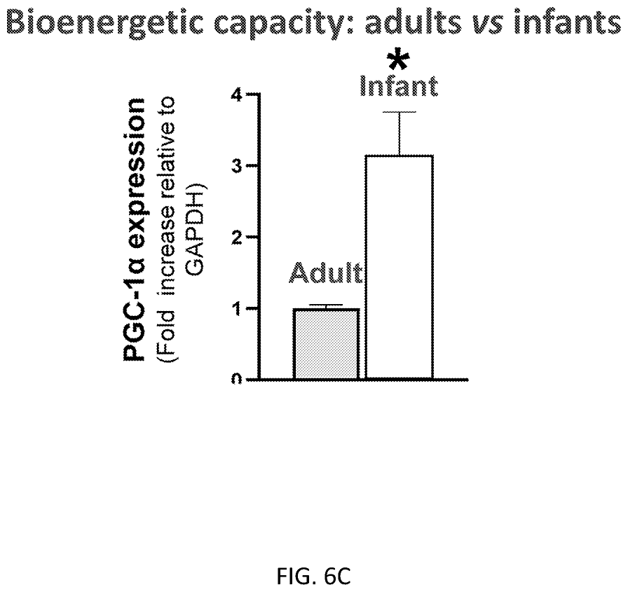

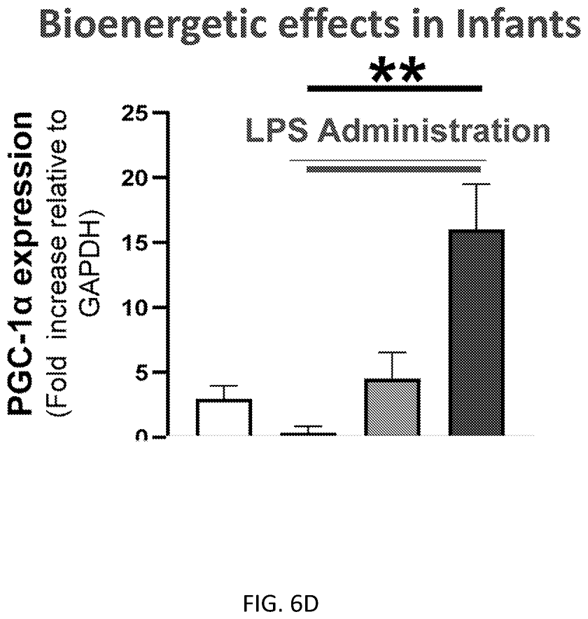

We quantified the number of immune cells (using FACS) present in the BAL of juvenile mice with ARDS following 24 h of LPS administration (FIG. 6A). The data shows increased neutrophil infiltration (CD11b+Ly6G+ cells) and pro-inflammatory M1 polarization of macrophages (M1/M2 ratio), which was not affected by saline or conventional steroid treatment, but which was significantly reduced with pFUS-UC-MSC-EVs delivered IV into mice. FIG. 6B shows that the effect on neutrophils and macrophages in juvenile mice is also dose dependent showing improved therapeutic efficacy at higher doses of pFUS-UC-MSC-EVs. In addition, (FIG. 6C) they have a greater bioenergetic capacity (as indexed by PGC-1α expression) within their lungs at baseline compared to adults, and (FIG. 6D) following inflammation this is completely depleted, but fully is restored with pFUS-UC-MSC-EVs. In summary, pFUS-UC-MSC-EVs offer a very promising clinically translatable therapeutic option to treat ARDS.

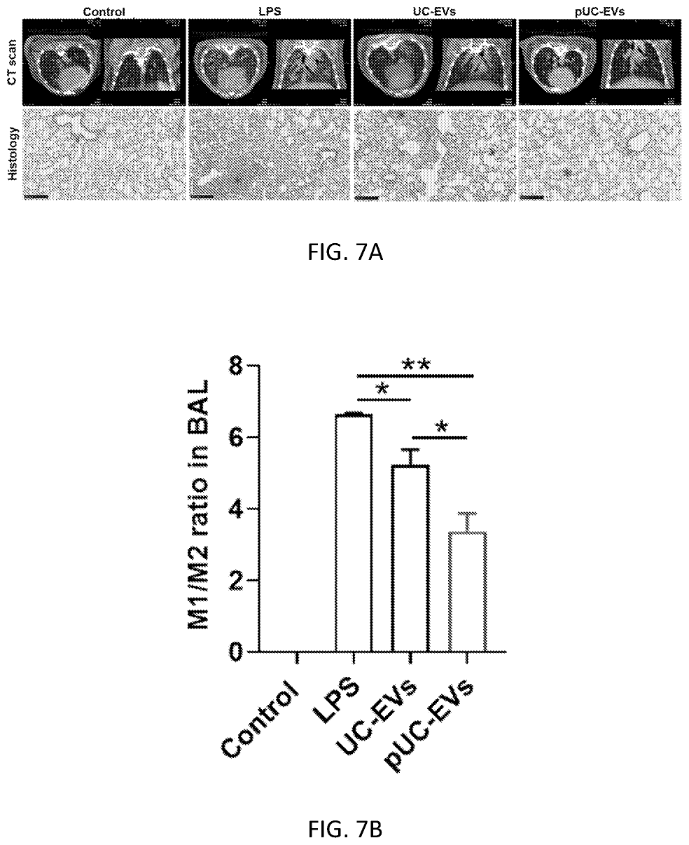

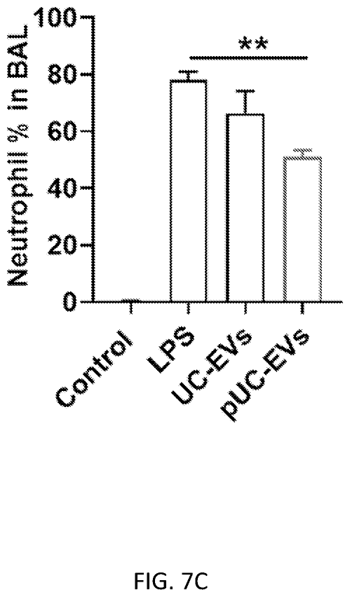

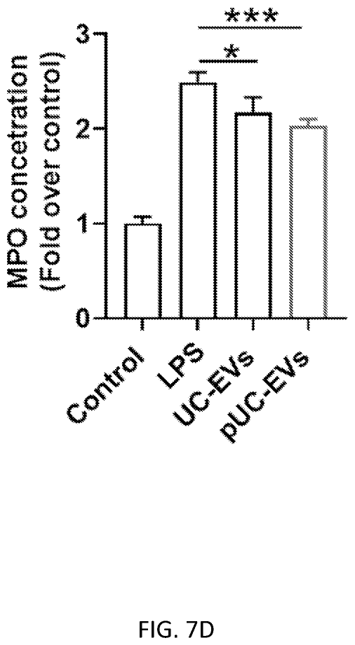

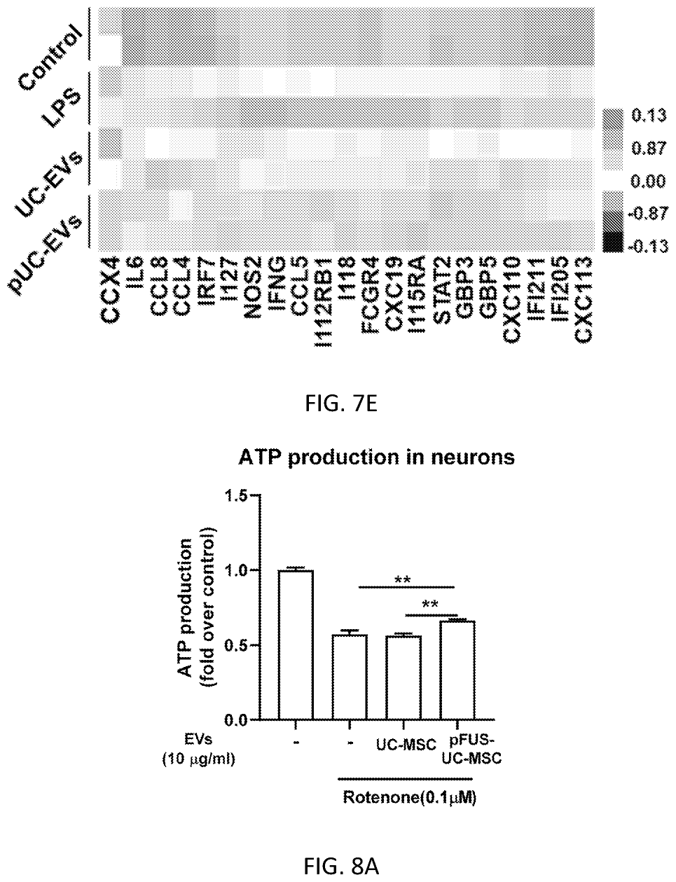

Pilot data showing intra-tracheal injection of pUC-EVs performed better than UC-EVs in reducing inflammation in ARDS in the lungs as indicated by (FIG. 7A) CT and H&E staining (*: inflammatory cells infiltration; $: Thickening of alveolar wall), (FIG. 7B) M1/M2 ratio in the BAL where UC-MSCs also inhibit pro-inflammatory M1 macrophage polarization while promoting anti-inflammatory M2 phenotypes (FIG. 7C) % of neutrophil in BAL and (FIG. 7D) neutrophil activity measure by MPO assay where the neutrophil infiltration and activation was significantly lower in the pUC-EVs treated groups. (FIG. 7E) The normalized counts from lung tissue transcriptome indicate changes in gene expression for genes related to inflammation where the decrease in the inflammation was observed in pUC-EVs treated groups. In summary, pUC-EVs improve the EVs therapy for the treatment of ARDS.

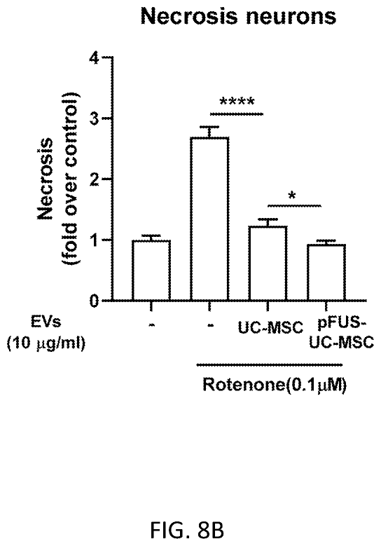

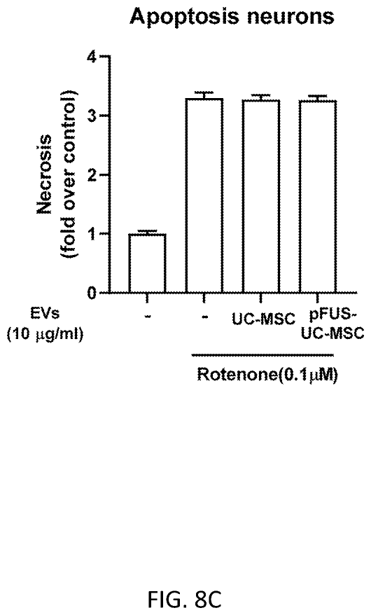

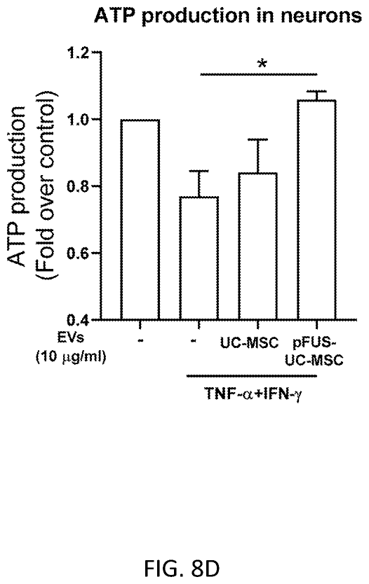

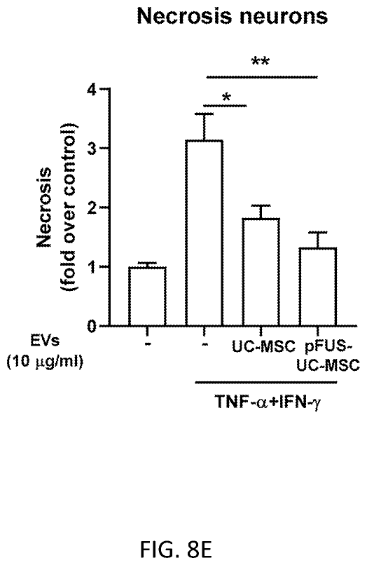

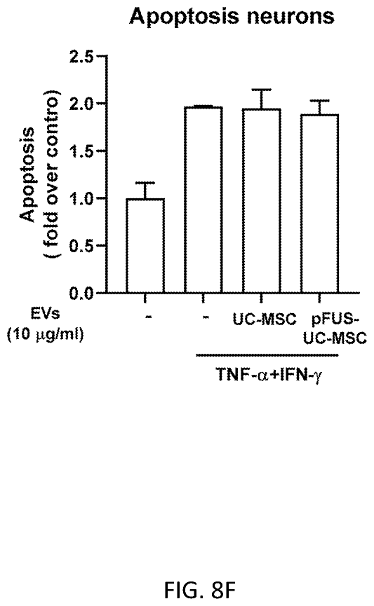

FIG. 8 shows an improvement of bioenergetic health and the viability of neurons with EVs therapy. FIGS. 8A and 8D show ATP production in neurons after treatment with a rotenone/inflammatory cocktail (TNF α and IFN-γ) where pFUS-UC-MSCs showed improvement in ATP generation compared to UC-MSC-EVs and reduced the cell death suggested by the decrease in necrosis (FIGS. 8B and 8E) without a change in apoptosis (FIGS. 8C and 8F).

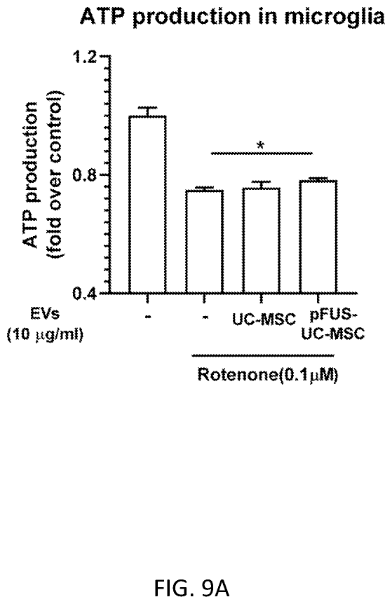

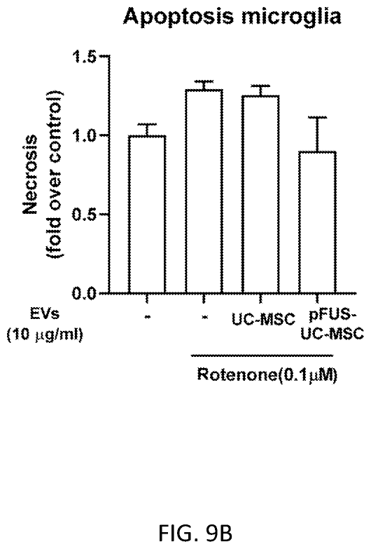

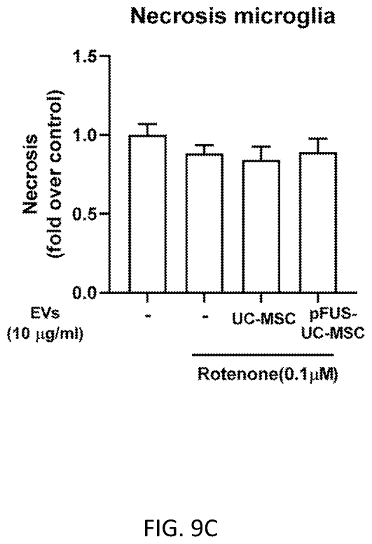

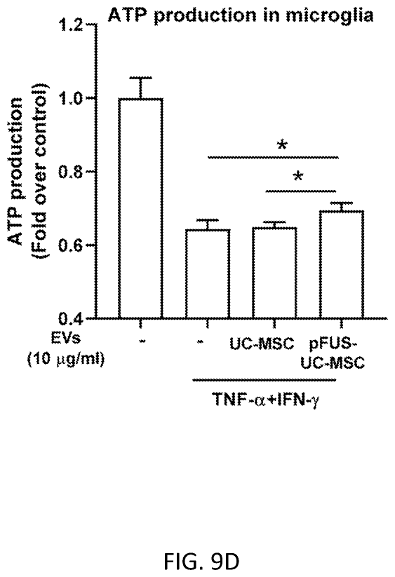

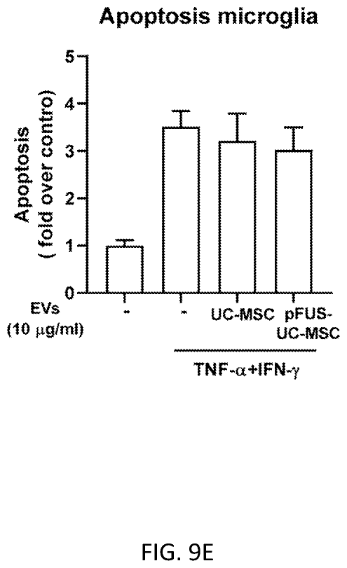

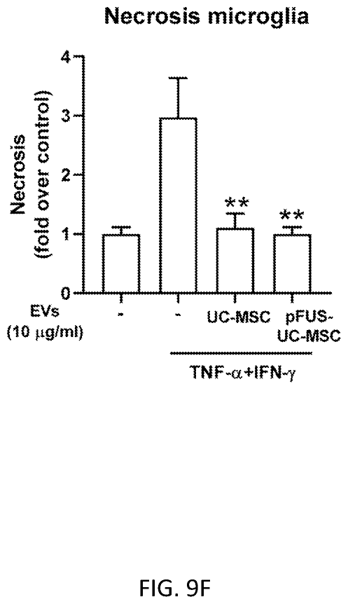

FIG. 9 shows an improvement of bioenergetic health and the viability of microglia with EVs therapy. FIGS. 9A and 9D show ATP production in neurons after rotenone/inflammatory cocktail (TNF α and IFN-γ) treatment where pFUS-UC-MSCs showed improvement in ATP generation and reduced the cell death suggested by the decrease in necrosis (FIGS. 9B and 9E) without a change in apoptosis (FIGS. 9C and 9F).

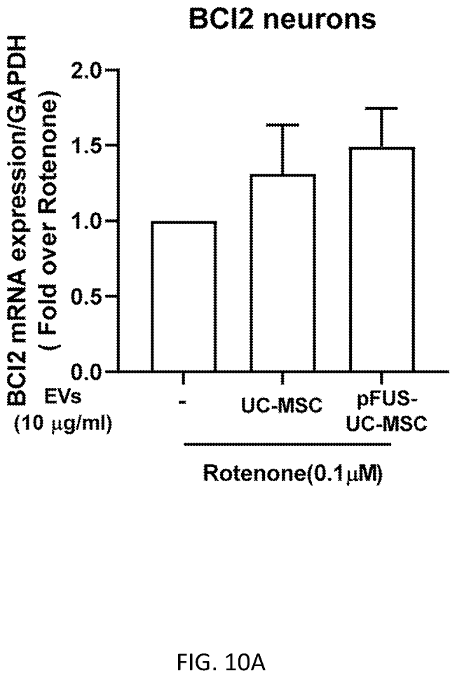

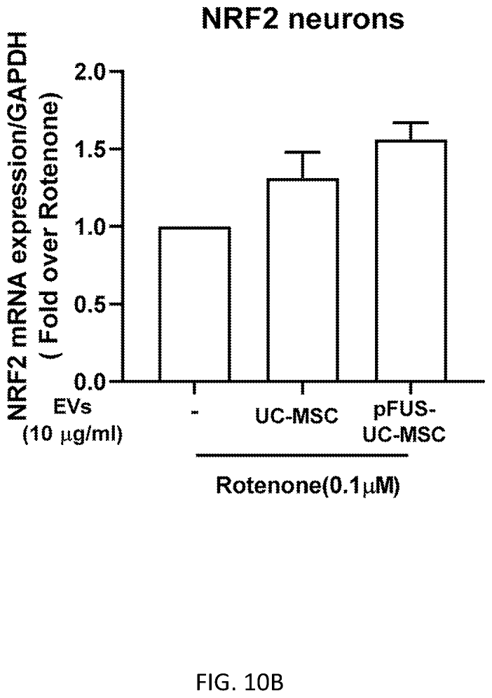

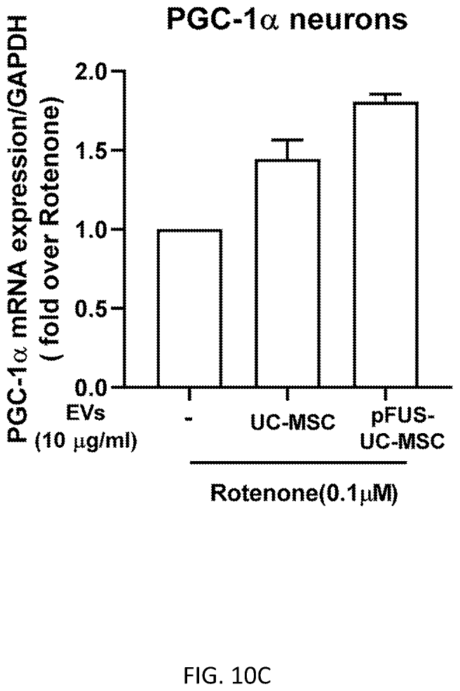

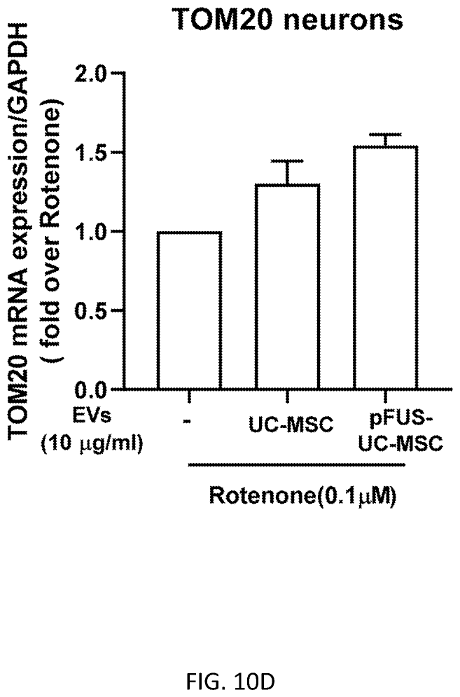

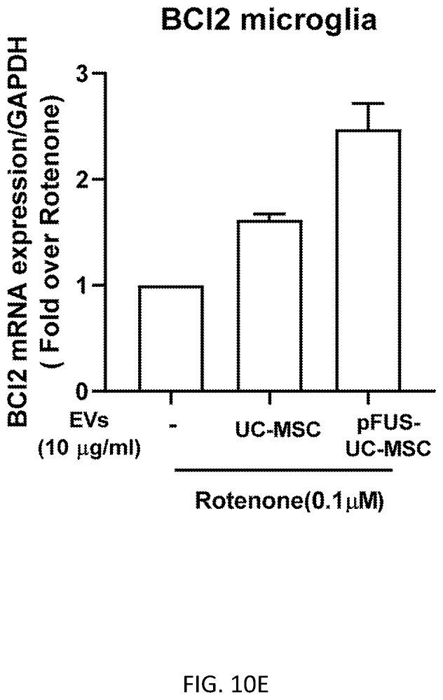

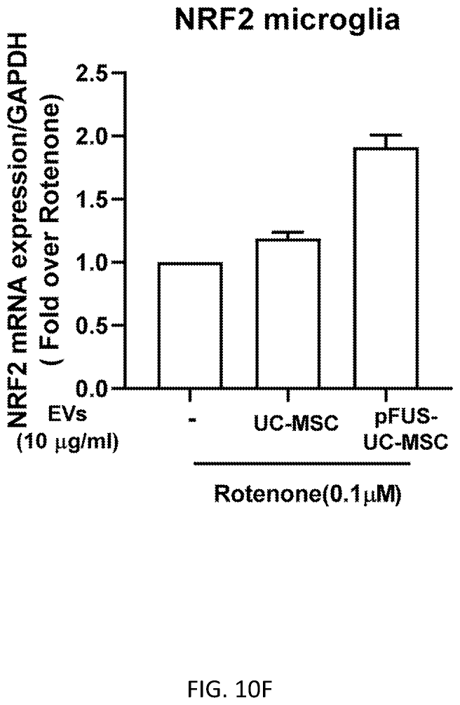

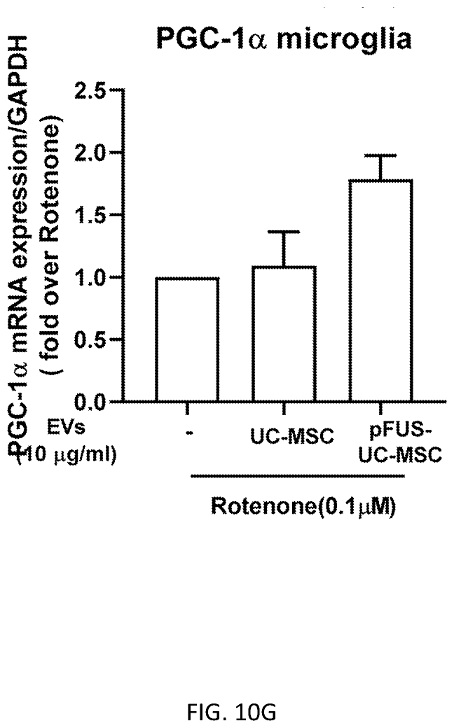

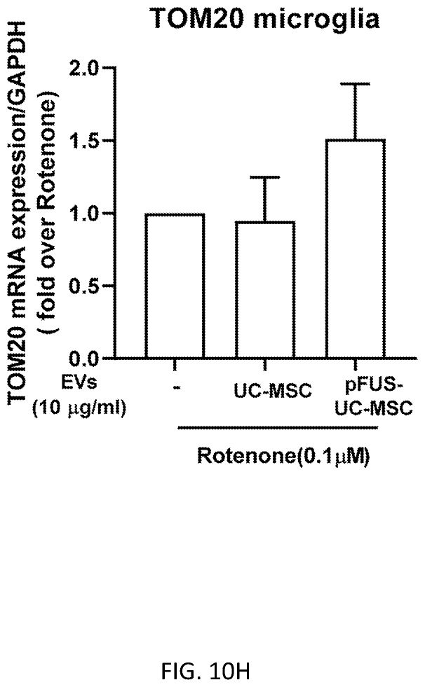

FIG. 10 shows an improvement of bioenergetic health and the viability of neurons and microglia with EV therapy. FIGS. 10A and 10E show BCL2 expression in neurons and microglia respectively where pFUS-UC-MSCs showed an increase in BCL2 expression suggesting a reduction in cell death. FIGS. 10B and 10F show NRF2 expression in neurons and microglia respectively indicating an increase in NRF2 expression suggesting a reduction in oxidative stress. FIG. 10 C and 10F show PGC-1^{α} expression in neurons and microglia respectively, and FIGS. 10D and 10H show TOM20 expression in neurons and microglia respectively suggesting mitochondrial biogenesis is occurring in neurons and microglia in response to treatment with pFUS-UC-MSC-EVs.

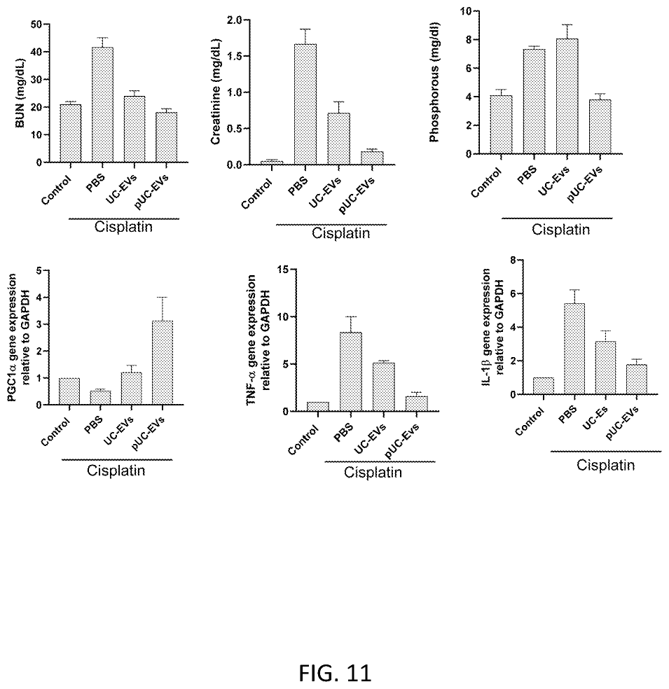

FIG. 11 shows the effects of pFUS UC-EVs in preventing cisplatin induced AKI. Intra-arterial delivery of pUC-EVs (100 ug/kg) in mouse was able to prevent an increase in BUN, sCreatinine. pUC-EVs also increased a marker for mitochondrial biogenesis (PGC1A), which may help in restoring bioenergetics in the kidney and reduce systemic inflammation (TNFα and IL1B).

### Conclusions

We have shown that MSC-EVs can be used as a therapy for ARDS by reducing lung injury, reducing inflammation, and modulating the immune system. Furthermore, we have shown MSCEVs can restore the bioenergetic health of injured cells and regulate the regeneration of the injured lung microenvironment through their cargo which contains mitochondrial-related products and miRNA. Hence, if MSC-EVs can mitigate the exudative phase of ARDS, and promote lung healing and regeneration, this will prevent or delay long-term complications such as reduced lung function and lung fibrosis. In addition we also observed improvement of bioenergetics of neuronal and microglial cells in our pilot experiments with pFUS-MSCs-EVs treatment. Furthermore, pFUS-MSCs-EVs also attenuate the acute kidney injury. We believe that pFUS-MSCs-EVs will also be useful for treatment of other inflammatory illnesses, age-related problems and illnesses linked to mitochondrial dysfunction.

### REFERENCES

1. Cloonan SM, Kim K, Esteves P, Trian T, Barnes PJ. Mitochondrial dysfunction in lung ageing and disease. European Respiratory Review. 2020;29(157).
2. Ryter SW, Choi AMJRb. Autophagy in lung disease pathogenesis and therapeutics. Redox Biol. 2015;4:215-25.

What is claimed is:

1. A method of treating a subject for a disease or condition associated with cellular-energy deficiency or mitochondrial dysfunction, the method comprising:
stimulating a mesenchymal stromal cell with sound waves; and
administering to the subject a therapeutically effective amount of extracellular vesicles derived from the mesenchymal stromal cell after said stimulating the mesenchymal stromal cell with the sound waves.

2. The method of claim 1, wherein said stimulating comprises administering an effective amount of the sound waves sufficient to increase levels in the extracellular vesicles of a mitochondrial microRNA (miRNA), a mitochondrial messenger RNA (mRNA), a mitochondrial protein, lipids, or a combination thereof, compared to the levels in extracellular vesicles produced by a reference mesenchymal stromal cell that is not stimulated with the sound waves.

3. The method of claim 2, wherein the mitochondrial miRNA, mRNA, or protein is involved in promoting mitochondrial biogenesis or production of adenosine triphosphate (ATP).

4. The method of claim 2 or 3, wherein the mitochondrial protein is COX-IV, TOM20, Complex I, Complex II, Complex II and Complex IV, citrate synthase, HSP60, PGC-1α, SIRT1, SIRT2, SIRT3, MFN, OPA1, DRP1, TRPC, PMCA, RhoA1, Miro1, or mtHSP70.

5. The method of any one of claims 2-4, wherein the mitochondrial miRNA regulates immunomodulation or metabolic health.

6. The method of claim 5, wherein the miRNA regulating immunomodulation is mir-9-5p, miR-15a-5p, miR-22-3p, miR-224-3p, miR-144-3p, or miR-146a-5p.

7. The method of claim 5, wherein the miRNA regulating metabolic health is miR-9-5p, miR-15a-5p, miR-16-5p, miR-18a-5p, miR-19b-3p, miR-20a-5p, miR-29a-3p, miR30a-5p, miR-30b-5p, miR-30e-5p, miR-34a-5p, miR-92a-3p, miR-142-3p, miR-146a-5p, or miR-148b-3p.

8. The method of any one of claims 1-7, wherein said stimulating comprises administering an effective amount of the sound waves sufficient to increase numbers of extracellular vesicles produced by the mesenchymal stromal cell compared to the numbers of the extracellular vesicles produced by a reference mesenchymal stromal cell that is not stimulated with the sound waves.

9. The method of any one of claims 1-8, wherein the mesenchymal stromal cell is from umbilical cord, placental tissue, adipose tissue, or bone marrow.

10. The method of any one of claims 1-9, wherein the extracellular vesicles are exosomes, microvesicles, apoptotic bodies, ectosomes, or microparticles.

11. The method of any one of claims 1-10, wherein the extracellular vesicles have diameters ranging from about 1 nm to 2000 nm.

12. £ The method of any one of claims 1-11, wherein the extracellular vesicles comprise one or more surface markers selected from the group consisting of TSG101, ALIX, CD63, and CD9.

13. The method of any one of claims 1-12, wherein the mesenchymal stromal cell is adherent or in a suspended population in culture.

14. The method of any one of claims 1-13, wherein the mesenchymal stromal cell is a genetically modified mesenchymal stromal cell.

15. The method of claim 14, wherein the extracellular vesicles derived from the genetically modified mesenchymal stromal cell after said stimulation with the sound waves comprise a short hairpin RNA (shRNA), a short interfering RNA (siRNA), a microRNA (miRNA), a Clustered Regularly Interspaced Short Palindromic Repeat (CRISPR) guide RNA, or a therapeutic peptide, polypeptide, or protein.

16. The method of any one of claims 1-15, further comprising lyophilizing the extracellular vesicles prior to administering to the subject.

17. The method of claim 16, wherein the extracellular vesicles are lyophilized in the presence of a surface-active stabilizer or cryoprotectant.

18. The method of any one of claims 1-17, wherein the extracellular vesicles are administered intravenously, intra-arterially, subcutaneously, percutaneously, intramuscularly, intrathecally, by pulmonary inhalation, or locally.

19. The method of any one of claims 1-18, wherein said stimulating comprises administering pulsed focused ultrasound (pFUS) to the mesenchymal stromal cell.

20. The method of claim 19, wherein the pFUS is administered to the mesenchymal stromal cell at an ultrasound frequency ranging from 20 kHz to 3.0 MHz with a pulse repetition frequency (PRF) ranging from 5 Hz to 200 Hz, a ultrasound duty cycle (DC) ranging from 0.1% to 50%, and a peak negative pressure (PNP) ranging from 0.1 MPa to 10 MPa.

21. The method of any one of claims 1-20, wherein a single cycle of treatment or multiple cycles of treatment are administered to the subject.

22. The method of any one of claims 1-21, wherein the disease or condition associated with cellular-energy deficiency or mitochondrial dysfunction is a mitochondrial disease, an inflammatory disease, a hereditary disease, an infection, a degenerative disease, a cardiovascular disease, aging, infarction, chronic fatigue syndrome, or cancer.

23. The method of any one of claims 1-22, wherein the disease associated with cellular-energy deficiency or mitochondrial dysfunction is a lung disease, kidney disease, or a neurodegenerative disease.

24. The method of claim 23, wherein the lung disease is chronic or acute respiratory distress syndrome (ARDS).

25. The method of claim 23, wherein the kidney disease is chronic or acute kidney injury (AKI).

26. The method of claim 23, wherein the neurodegenerative disease is Alzheimer's disease.

27. The method of any one of claims 1-26, wherein the extracellular vesicles are administered with a single route of administration or multiple routes of administration.

28. The method of any one of claims 1-27, further comprising imaging damaged tissue before, during, or after said administering the extracellular vesicles.

29. The method of claim 28, wherein said imaging is performed by ultrasound, magnetic resonance imaging (MRI), computed tomography (CT), or scintigraphy.

30. The method of any one of claims 1-29, further comprising coculturing the extracellular vesicles with the mesenchymal stromal cell or another type of cell prior to said administering the extracellular vesicles to the subject.

31. The method of any one of claims 1-30, further comprising administering a cellular therapy to the subject.

32. A composition comprising extracellular vesicles derived from a mesenchymal stromal cell that has been stimulated with sound waves for use in a method of treating a disease or condition associated with cellular-energy deficiency or mitochondrial dysfunction.

33. The composition of claim 32, wherein the mesenchymal stromal cell has been stimulated with sound waves by administering pulsed focused ultrasound (pFUS) to the mesenchymal stromal cell.

34. The composition of claim 33, wherein the pFUS has been administered to the mesenchymal stromal cell at an ultrasound frequency ranging from 20 kHz to 3.0 MHz with a pulse repetition frequency (PRF) ranging from 5 Hz to 200 Hz, a ultrasound duty cycle (DC) ranging from 0.1% to 50%, and a peak negative pressure (PNP) ranging from 0.1 MPa to 10 MPa.

35. The composition of any one of claims 32-34, wherein the disease associated with cellular-energy deficiency or mitochondrial dysfunction is a lung disease, kidney disease, or a neurodegenerative disease.

36. The composition of claim 35, wherein the lung disease is chronic or acute respiratory distress syndrome (ARDS).

37. The composition of claim 35, wherein the kidney disease is chronic or acute kidney injury (AKI).

38. The composition of claim 35, wherein the neurodegenerative disease is Alzheimer's disease.

39. The composition of any one of claims 32-38, further comprising a pharmaceutically acceptable excipient.

40. A method of improving metabolic health of a damaged, exhausted, or diseased cell, the method comprising:
stimulating a mesenchymal stromal cell with sound waves;
collecting extracellular vesicles secreted from the mesenchymal stromal cell after said stimulating the mesenchymal stromal cell with the sound waves;
contacting the damaged, exhausted, or diseased cell with an effective amount of the extracellular vesicles, wherein the metabolic health of the damaged, exhausted, or diseased cell is improved.

41. The method of claim 40, wherein said contacting is performed in vivo or ex vivo.

42. The method of claim 40, further comprising culturing the damaged, exhausted, or diseased cell in the presence of the extracellular vesicles.

43. The method of any one of claims 40-42, wherein the damaged, exhausted, or diseased cell is an immune cell, an epithelial cell, or an endothelial cell.

44. The method of claim 43, wherein the immune cell is a macrophage, a dendritic cell, a T cell, a B cell, a natural killer cell, or a monocyte.

45. The method of claim 44, wherein the T cell is an exhausted T cell.

46. The method of any one of claims 40-45, further comprising performing cellular therapy with the damaged, exhausted, or diseased cell after the metabolic health of the damaged, exhausted, or diseased cell is improved from said contacting the damaged, exhausted, or diseased cell with the extracellular vesicles.
